(19) United States
(12) Patent Application Publication
(10) Pub. No.: US 2026/0162763 A1
(43) Pub. Date: Jun. 11, 2026

(54) AUTOMATED LIGAND DESIGN

(71) Applicant: Isomorphic Labs Limited, London (GB)

(72) Inventors: Daniele Grattarola, London (GB); Christoph Johann Feinauer, London (GB); Maxwell Elliot Jaderberg, London (GB)

(21) Appl. No.: 19/294,856

(22) Filed: Aug. 8, 2025

Related U.S. Application Data
(60) Provisional application No. 63/682,200, filed on Aug. 12, 2024.

(51) Int. Cl. G16B 15/30; G16B 40/30; G16B 45/00; G16B 50/30

(52) U.S. Cl. CPC G16B 15/30; G16B 40/30; G16B 45/00; G16B 50/30

(57) ABSTRACT

Methods, systems, and apparatus, including computer programs encoded on a computer storage medium, for iteratively designing a ligand for binding to a protein. In one aspect, a method comprises, at each of a plurality of design iterations, performing operations comprising: generating, by a controller, a current set of ligand design criteria that define desired characteristics of the ligand; generating, using a generative model and while the generative model is conditioned on: (i) protein data characterizing the protein, and (ii) the ligand design criteria generated by the controller, data defining a set of candidate ligands that are predicted to bind to the protein; determining, for each of a plurality of the candidate ligands, a respective quality score for the candidate ligand based on properties of the candidate ligand; and updating the controller using the respective quality scores for the candidate ligands.

### CROSS-REFERENCE TO RELATED APPLICATION

This application claims priority under 35 U.S.C. 119 to Provisional Application No. 63/682,200, filed Aug. 12, 2024, which is incorporated by reference.

### BACKGROUND

This specification relates to computationally designing a ligand for binding to a protein.

A “ligand” can refer to a molecule or compound that binds to a target molecule, e.g., a protein. Ligands can include, e.g., small molecules, proteins, or nucleic acids (e.g., ribonucleic acids (RNA) or deoxyribonucleic acids (DNA)).

A “protein” can refer to any biological molecule that is specified by one or more sequences (or “chains”) of amino acids. For example, the term protein can refer to a protein domain, e.g., a portion of an amino acid chain of a protein that can undergo protein folding nearly independently of the rest of the protein. As another example, the term protein can refer to a protein complex, i.e., that includes multiple amino acid chains that jointly fold into a protein structure.

### SUMMARY

This specification describes a system implemented as computer programs on one or more computers in one or more locations that can computationally design a ligand for binding to a protein.

A “small molecule” can refer to a low molecular weight organic compound, e.g., less than 900 Daltons.

A “multiple sequence alignment” (MSA) for an amino acid chain in a protein specifies a sequence alignment of the amino acid chain with multiple additional amino acid chains, e.g., from other proteins, e.g., homologous proteins. More specifically, the MSA can define a correspondence between the positions in the amino acid chain and corresponding positions in multiple additional amino acid chains. A MSA for an amino acid chain can be generated, e.g., by processing a database of amino acid chains using any appropriate computational sequence alignment technique, e.g., progressive alignment construction. The amino acid chains in the MSA can be understood as having an evolutionary relationship, e.g., where each amino acid chain in the MSA may share a common ancestor. The correlations between the amino acids in the amino acid chains in a MSA for an amino acid chain can encode information that is relevant to predicting the structure of the amino acid chain.

A “binding pocket” on a protein can refer to a specific three-dimensional cavity or crevice within the structure of the protein where a ligand can bind to the protein. The binding pocket can, in some cases, be understood as a “lock” that fits the shape and chemical properties of ligands that act as “keys” for the lock. In other cases, the ligand may initially not fit perfectly into the binding pocket, e.g., due to structural differences or slight mismatches in shape or chemical groups, but conformational changes during binding can cause the interaction between the ligand and the binding pocket to become more complementary and specific, e.g., as in induced-fit binding. Examples of binding pockets include, e.g., orthosteric binding pockets, allosteric binding pockets, and cryptic binding pockets.

A “template” protein for a given protein can refer to a protein that is “similar” to the given protein, e.g., such that the value of a similarity measure between the template protein and the given protein satisfies (e.g., exceeds) a threshold (e.g., 0.8, or 0.9, or 0.99, or any other appropriate threshold). Similarity between a first protein and a second protein can be measured using any appropriate similarity measure, e.g., a sequence identity or percent identity similarity measure between the respective amino acid sequence(s) of the first protein and the second protein.

A first neural network can be referred to as a “subnetwork” of a second neural network if the first neural network is included in the second neural network.

A “block” (e.g., a “self-attention block”) in a neural network can refer to a group of one or more neural network layers in the neural network.

An “embedding” of an entity (e.g., an atom, or a ligand, or a protein) can refer to a representation of the entity as an ordered collection of numerical values, e.g., a vector, matrix, or other tensor of numerical values.

“Conditioning” a model (e.g., a generative model) or a neural network (e.g., a denoising neural network) or an operation (e.g., a self-attention operation) on conditioning data (e.g., an embedding representing a protein or a set of ligand design criteria) can refer to providing the conditioning data (or a representation, e.g., an embedding, of the conditioning data) as an input (e.g., a side input) to the model, neural network, or operation, such that outputs generated by the model, neural network, or operation are influenced by (depend on) the conditioning data.

A “binding affinity” of a ligand for a protein refers to the strength or degree of attraction between the ligand and the protein when they interact to form a complex. For example, a binding affinity may be indicative of an energy of formation of the complex, such as a Gibbs free energy of formation.

A 3D spatial position of an atom can be represented by a set of coordinates in an appropriate coordinate system, e.g., a 3D Cartesian coordinate system or a spherical coordinate system.

“Absorption” properties of a molecule (e.g., a ligand) characterize how well the molecule is absorbed in a subject following administration, and can include one or more of: a solubility of the molecule, a permeability of the molecule (e.g., across a cell membrane), a chemical stability of the molecule, and so forth.

“Distribution” properties of a molecule (e.g., a ligand) characterize how the molecule spreads through the body of the subject after being administered to the subject, and can include one or more of: a lipophilicity of the molecule, a strength of plasma protein binding of the molecule, a volume of distribution of the molecule (a measure of the extent to which the molecule spreads into body tissues), and so forth.

“Metabolism” properties of a molecule (e.g., a ligand) characterize how the molecule is chemically transformed in the body, and can include one or more of: properties characterizing which enzymatic pathways are responsible for metabolizing the molecule, metabolic rate properties, properties characterizing the activity of metabolites that are generated by metabolism of the molecule, and so forth.

“Excretion” properties of a molecule (e.g., a ligand) characterize the mechanisms and pathways through which the molecule and its metabolites are removed from the body, and can characterize whether the molecule is excreted through renal excretion, or hepatic excretion, or pulmonary excretion, and so forth.

“Toxicity” properties of a molecule (e.g., a ligand) characterize the potential of the molecule to cause harmful effects to a subject, and can include one or more of: acute toxicity properties, chronic toxicity properties, carcinogenicity properties, organ toxicity properties, reproductive toxicity properties, developmental toxicity properties, and so forth.

A “computing unit” may be, e.g., a computer, a core within a computer having multiple cores, or other hardware or software, e.g., a dedicated thread, within a computer capable of independently perform operations. Computing units may include processor cores, processors, microprocessors, special-purpose logic circuitry, e.g., an FPGA (field-programmable gate array) or an ASIC (application-specific integrated circuit), or any other appropriate computing units. In some examples, the computing units are all the same type of computing unit. In other examples, the computing units may be different types of computing units. For example, one computing unit may be a CPU while other computing units may be GPUs.

According to one aspect there is provided a method performed by one or more computers, the method comprising: iteratively designing a ligand for binding to a protein by, at each of a plurality of design iterations, performing operations comprising: generating, by a controller, a current set of ligand design criteria that define desired characteristics of the ligand; generating, using a generative model and while the generative model is conditioned on: (i) protein data characterizing the protein, and (ii) the ligand design criteria generated by the controller, data defining a set of candidate ligands that are predicted to bind to the protein; determining, for each of a plurality of the candidate ligands, a respective quality score for the candidate ligand based on properties of the candidate ligand; and updating the controller using the respective quality scores for the candidate ligands.

In some implementations, at each of one or more design iterations, updating the controller using the respective quality scores for the candidate ligands comprises: updating the controller to encourage the controller to generate ligand design criteria that result in generation of candidate ligands with improved quality scores at subsequent design iterations.

In some implementations, at each of one or more design iterations after a first design iteration, generating, by the controller, the current set of ligand design criteria that define desired characteristics of the ligand comprises: generating the current set of ligand design criteria based at least in part on quality scores for candidate ligands generated at preceding design iterations.

In some implementations, over the plurality of design iterations, the controller performs a directed search through a space of possible ligand design criteria to optimize quality scores of candidate ligands generated in accordance with the ligand design criteria.

In some implementations, the controller is implemented using a policy neural network; and at each of one or more design iterations, generating, by the controller, the current set of ligand design criteria that define desired characteristics of the ligand comprises: generating the current set of ligand design criteria as an output of the policy neural network.

In some implementations, at each of one or more design iterations after a first design iteration, generating the current set of ligand design criteria as an output of the policy neural network comprises: generating a network input to the policy neural network that includes data representing candidate ligands generated at one or more preceding design iterations; and processing the network input using the policy neural network to generate the current set of ligand design criteria.

In some implementations, the network input to the policy neural network comprises, for each candidate ligand selected for inclusion in the network input to the policy neural network, data representing one or more of: a three-dimensional (3D) structure of the candidate ligand; a 3D structure of a complex that includes the protein and the candidate ligand; or a quality score for the candidate ligand.

In some implementations, generating the network input to the policy neural network comprises selecting a plurality of previously generated candidate ligands for inclusion in the network input by performing operations comprising: applying a clustering operation to a set of previously generated candidate ligands to identify a set of candidate ligand clusters; and selecting a plurality of previously generated candidate ligands for inclusion in the network input based on the set of candidate ligand clusters.

In some implementations, updating the controller using the respective quality scores for the candidate ligands comprises: training the policy neural network, by a reinforcement learning training technique, using rewards that are based on the quality scores for the candidate ligands.

In some implementations, the controller is implemented using a genetic algorithm; and at each of one or more design iterations, generating, by the controller, the current set of ligand design criteria that define desired characteristics of the ligand comprises: obtaining data defining a current population of ligand design tensors, wherein each ligand design tensor specifies a respective set of ligand design criteria and is associated with an overall quality score that is derived from quality scores of candidate ligands generated in accordance with the set of ligand design criteria; selecting a set of at least two parent ligand design tensors from the current population of ligand design tensors based on the overall quality scores of the ligand design tensors; and combining two or more of the parent ligand design tensors to generate a child ligand design tensor that defines the current set of ligand design criteria.

In some implementations, at each of one or more design iterations, updating the controller using the quality scores for the candidate ligands comprises: determining an overall quality score for the child ligand design tensor based on the quality scores for the candidate ligands generated based on the child ligand design tensor; and augmenting the current population of ligand design tensors to include data defining the child ligand design tensor and the overall quality score for the child ligand design tensor.

In some implementations, the controller is implemented using a simulated annealing algorithm; and at each of one or more design iterations, generating, by the controller, the current set of ligand design criteria that define desired characteristics of the ligand comprises: obtaining data defining a current ligand design tensor that defines a set of ligand design criteria; and generating a candidate ligand design tensor by stochastically modifying the current ligand design tensor, wherein the candidate ligand design tensor defines the current set of ligand design criteria.

In some implementations, at each of one or more design iterations, updating the controller using the respective quality scores for the candidate ligands comprises: determining an overall quality score for the candidate ligand design tensor based on the quality scores for the candidate ligands generated based on the candidate ligand design tensor; and determining whether to update the current ligand design tensor to the candidate ligand design tensor using: (i) a current temperature parameter, and (ii) the overall quality score for the candidate ligand design tensor; wherein the current temperature parameter at the design iteration is determined based on a predefined schedule.

In some implementations, the method further comprises, at each of one or more design iterations, determining a measure of diversity of the set of candidate ligands generated at the design iteration; wherein the controller is updated at the design iteration based at least in part on the measure of diversity of the set of candidate ligands generated at the design iteration.

In some implementations, the method further comprises, at each of one or more design iterations: generating a plurality of new candidate ligands by fragmenting and reassembling candidate ligands from the set of candidate ligands generated by the generative model at the design iteration; and augmenting the set of candidate ligands by adding the plurality of new candidate ligands to the set of candidate ligands.

In some implementations, the method further comprises, at each of one or more design iterations, generating candidate ligands using the generative model until a termination criterion is satisfied; wherein the termination criterion is satisfied when a rate of increase in a measure of diversity of the set of candidate ligands generated at the design iteration fails to satisfy a threshold.

In some implementations, at each of one or more design iterations, the current set of ligand design criteria define a respective target value for each of one or more global properties of the ligand that characterize the ligand as a whole.

In some implementations, at each of one or more design iterations, the current set of ligand design criteria define a respective target value for a set of global properties of the ligand characterizing one or more of: a binding affinity of the ligand for the protein, absorption properties of the ligand, distribution properties of the ligand, metabolism properties of the ligand, excretion properties of the ligand, toxicity properties of the ligand, a number of rings in the ligand, a molecular weight of the ligand, a lipophilicity of the ligand, an ability of the ligand to donate or accept hydrogen bonds, a total polar surface area of the ligand, a number of rotatable bonds in the ligand, a number of chiral centers in the ligand, a number of electrophilic centers in the ligand, a number of nucleophilic centers in the ligand.

In some implementations, at each of one or more design iterations, the current set of ligand design criteria define a respective target value for each of one or more atom-specific properties of the ligand that each relate to a specific atom in the ligand.

In some implementations, at each of one or more design iterations, the current set of ligand design criteria define a respective target value for a set of atom-specific properties of the ligand that, for each of one or more atoms in the ligand, characterize one or more of: an elemental type of the atom, a hybridization state of the atom, or a partial charge of the atom.

In some implementations, at each of one or more design iterations, the current set of ligand design criteria comprises ligand scaffolding data that defines a respective target three-dimensional (3D) spatial position of each of one or more atoms in the ligand.

In some implementations, the generative model is implemented using a generative diffusion model, or using a generative adversarial neural network, or using a normalizing flow model.

In some implementations, at each of one or more design iterations, generating, using the generative model and while the generative model is conditioned on: (i) protein data characterizing the protein, and (ii) the ligand design criteria generated by the controller, data defining the set of candidate ligands that are predicted to bind to the protein comprises: generating each candidate ligand by sampling from a distribution over a space of possible ligands, wherein the distribution over the space of possible ligands depends on: (i) the protein data characterizing the protein, and (ii) the ligand design criteria generated by the controller.

In some implementations, at each of one or more design iterations, determining, for each of the plurality of candidate ligands, the respective quality score for the candidate ligand based on properties of the candidate ligand comprises, for each candidate ligand: processing a model input that characterizes the candidate ligand using a property prediction machine learning model to generate a model output that defines one or more predicted properties of the candidate ligand.

In some implementations, the model input to the property prediction machine learning model comprises a predicted 3D structure of the candidate ligand.

In some implementations, the model input to the property prediction machine learning model comprises a predicted 3D structure of a complex that includes the protein and the candidate ligand.

In some implementations, at each of one or more design iterations, for each of one or more candidate ligands, the quality score for the candidate ligand is based on one or more of: a binding affinity of the candidate ligand for the protein, absorption properties of the candidate ligand, distribution properties of the candidate ligand, metabolism properties of the candidate ligand, excretion properties of the candidate ligand, toxicity properties of the candidate ligand, a number of rings in the candidate ligand, a molecular weight of the candidate ligand, a lipophilicity of the candidate ligand, an ability of the candidate ligand to donate or accept hydrogen bonds, a total polar surface area of the candidate ligand, a number of rotatable bonds in the candidate ligand, a number of chiral centers in the candidate ligand, a number of electrophilic centers in the candidate ligand, or a number of nucleophilic centers in the candidate ligand.

In some implementations, the method further comprises physically synthesizing one or more of the candidate ligands.

According to another aspect, there is provided a method comprising: generating a collection of ligands for a protein using the methods described herein; determining, for each ligand in the collection of ligands, one or more respective properties of the ligand; and selecting one or more ligands in the collection of ligands for physical synthesis based at least in part on the properties of the ligands.

In some implementations, the method further comprises physically synthesizing the one or more selected ligands.

According to another aspect, there is provided a method of obtaining a ligand, wherein the ligand is a drug or a ligand of an industrial enzyme, the method comprising: performing the methods described herein to determine a plurality of candidate ligands for a target protein; and evaluating a respective interaction of each candidate ligand with the target protein; and selecting one or more of the candidate ligands dependent on a result of the evaluating.

In some implementations, the target protein comprises a receptor or enzyme, and wherein each candidate ligand is an agonist or antagonist of the receptor or enzyme.

In some implementations, the target protein comprises an antibody or aptamer target, in particular a virus or cancer cell protein, and wherein the ligand binds to the antibody or aptamer target to provide a therapeutic effect.

According to another aspect, there is provided a system comprising: one or more computers; and one or more storage devices communicatively coupled to the one or more computers, wherein the one or more storage devices store instructions that, when executed by the one or more computers, cause the one or more computers to perform operations of the methods described herein.

According to another aspect, there are provided one or more non-transitory computer storage media storing instructions that when executed by one or more computers cause the one or more computers to perform operations of the methods described herein.

Particular embodiments of the subject matter described in this specification can be implemented so as to realize one or more of the following advantages.

Drug discovery can involve identifying specific molecules within the body that are involved in a disease process. These molecules are often proteins, such as enzymes, receptors, or signaling proteins, that play a key role in the disease's development or progression. A ligand, often a small molecule, peptide, or antibody, can be selected to bind specifically to an identified target protein and modify its biological activity. When a drug that includes the ligand is administered to a patient, the ligand can bind to the target protein with high affinity and in doing so contribute to achieving a therapeutic effect in the patient. For instance, if the target protein is an enzyme involved in a disease process, the ligand can inhibit its activity, thus disrupting the disease pathway. More generally, the interaction between the ligand and the target protein can activate, inhibit, or alter the function of the target protein to achieve a therapeutic effect. The ligand can, for example, be an agonist or antagonist of a receptor of the target protein. Therefore, identifying ligands with (relatively) high (or low) binding affinity for proteins can be an important step in the process of drug discovery, and also in other areas such as pest or pathogen control in agriculture, modifying plant species to improve growth and stress resistance, and so forth, as will be described in more detail later in this specification.

One approach to identifying a ligand for binding to a protein involves using a generative model that, when conditioned on: (i) protein data characterizing the protein, and (ii) a set of ligand design criteria defining desired characteristics of the ligand, can generate candidate ligands for the protein. The generative model can be configured through training by a machine learning training technique to generate candidate ligands that bind to the protein and that satisfy the ligand design criteria. Ligand design criteria can specify, e.g.: a respective target (desired) value for each of one or more properties of the ligand (e.g., physio-chemical or biochemical properties), or ligand scaffolding data, or both. The target ligand properties can specify a target (desired) value for any appropriate “global” or “atom-specific” properties of the ligand. Ligand scaffolding data can specify at least a portion of the chemical structure of the ligand.

Using a generative model (as described above) for designing a ligand for binding to a protein requires specifying ligand design criteria for use in conditioning the generative model. However, specifying ligand design criteria that result in the generation of high performing, e.g., strongly binding, candidate ligands is a complex and challenging task, e.g., for the reasons set forth next.

First, the space of possible ligand design criteria can be a high-dimensional space (e.g., with tens or hundreds of dimensions). Selecting a set of ligand design criteria that are mutually compatible and that result in the generation of high-performing candidate ligands can require selecting properties that range from atom-specific (e.g., elemental types or hybridization states of particular atoms in the ligand) to global (e.g., the number of rings in the ligand or the absorption properties of the ligand) to geometric (e.g., the 3D structure of parts of the ligand). Further, the approach to selecting values for different ligand design criteria may be highly dependent upon the target protein, and any heuristic rules developed for selecting ligand design criteria for one target protein may not extend to any other target proteins.

Second, in some cases, candidate ligands generated by the generative model may satisfy certain ligand design criteria only approximately, or may entirely fail to satisfy certain ligand design criteria. This may occur, for instance, if the ligand design criteria are mutually incompatible (e.g., if there does not exist a ligand that binds to the protein that simultaneously satisfies all the ligand design criteria), or if the generative model parameters have not been trained on a sufficient amount or type of training data to enable the precise generation of candidate ligands that bind to the protein and that satisfy all the ligand design criteria. Thus any approach for selecting ligand design criteria must account for additional uncertainty in the behavior of the generative model, e.g., the uncertainty in whether (and to what degree) the generative model can generate candidate ligands that satisfy the ligand design criteria.

Third, the process of designing a ligand can require many iterations. More specifically, one choice of ligand design criteria can be used to generate many thousands of candidate ligands, many (or all) of which may be ultimately unsuitable, but some of which may have certain desirable properties, or which may suggest avenues for further exploration. However, selecting ligand design criteria for conditioning the generative model is a challenging task in the first instance (as outlined above), and this difficulty is compounded when determining how to modify initial choices of ligand design criteria in order to facilitate exploration of promising regions in the chemical space of possible ligands.

The system described in this specification addresses these issues and enables rapid and systematic design of high-performing ligands. The system uses a controller to perform an automated, directed search through the space of possible ligand design criteria in order to optimize quality scores characterizing the performance of ligands generated based on the ligand design criteria.

More specifically, at each design iteration in a sequence of design iterations, the system uses the controller to generate a current set of ligand design criteria at the design iteration. The system conditions the generative model on the current set of ligand design criteria, and then generates a set of candidate ligands. The system determines a respective quality score for each candidate ligand that characterizes a suitability of the candidate ligand for a downstream task (e.g., use in a drug) based on properties of the candidate ligand. The system then updates the controller based on the quality scores for the candidate ligands, in particular, to encourage (e.g., bias) the controller to generate ligand design criteria that lead to candidate ligands with improved quality scores at subsequent design iterations.

At each design iteration after the first design iteration, the controller generates the ligand design criteria in a manner that is based (either directly or indirectly) on the quality scores of candidate ligands generated using ligand design criteria selected (generated) at previous design iterations. Thus, for some or all of the design iterations after the first design iteration, the controller leverages information about the quality scores of candidate ligands generated at previous design iterations in order to inform the generation of the ligand design criteria at the current design iteration.

The system addresses the challenges of designing ligands using a generative model, as outlined above. In particular, the system can implement the controller, e.g., using a policy neural network, or a genetic algorithm, or a simulated annealing algorithm, that allows for automated generation of effective ligand design criteria, even when the space of ligand design criteria is high-dimensional. Further, the controller can treat the generative model as a “black box,” i.e., without making assumptions about the operations of the generative model, and can adaptively account for uncertainty in the behavior of the generative model. Further, the controller is configured to refine the ligand design criteria over the sequence of design iterations in a manner that optimizes the performance of candidate ligands generated by the generative model based on the ligand design criteria.

The system can reduce consumption of computational resources during ligand design. More specifically, the system can leverage a controller to perform an efficient and directed search through the space of possible ligand design criteria and can converge upon ligand design criteria that result in the generation of high-performing candidate ligands over fewer design iterations than would be required, e.g., by an approach that uses manual or heuristic techniques to select ligand design criteria. Each design iteration can be computationally intensive, e.g., as the generative model generates large numbers (e.g., thousands or millions) of candidate ligands, some or all of which are screened using property prediction machine learning models, computational chemistry calculations, and so forth. Thus by reducing the number of required design iterations, the system can dramatically reduce consumption of computational resources (e.g., memory and computing power) during computational ligand design.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example ligand design system.

FIG. 2 is a flow diagram of an example process for iteratively designing a ligand for binding to a protein.

FIG. 3 is a flow diagram of an example process for determining quality scores for a set of candidate ligands generated at a current design iteration.

FIG. 4 is a flow diagram of an example process for generating ligand design criteria using a controller implemented as a policy neural network.

FIG. 5 is a flow diagram of an example process for generating ligand design criteria using a controller implemented using a genetic algorithm.

FIG. 6 is a flow diagram of an example process for generating ligand design criteria using a controller implemented using a simulated annealing algorithm.

FIG. 7A shows an example embedding neural network that can generate conditioning data for conditioning the generative model that generates candidate ligands.

FIG. 7B is a flow diagram of an example process for processing ligand design criteria using a design embedding neural network to generate a design embedding.

FIG. 7C provides an illustration of a collection of initial atom embeddings generated by an embedding block of a design embedding neural network.

FIG. 8A is a flow diagram of an example process for processing a protein embedding of a protein and a design embedding of ligand design criteria using a fusion neural network to generate latent conditioning data that jointly represents the protein and the ligand design criteria.

FIG. 8B illustrates operations performed by the ligand design system to generate the latent conditioning data.

FIG. 9 is a flow diagram of an example process for generating data defining a ligand that is predicted to bind to a protein using a generative diffusion model that includes a denoising neural network.

FIG. 10 is a flow diagram of an example process for generating a denoising output using a denoising neural network conditioned on latent conditioning data.

FIG. 11 is a flow diagram of an example process for updating a set of current atom embeddings using a self-attention operation that is implemented by a self-attention block of the denoising neural network and that is conditioned on the latent conditioning data.

FIG. 12 is a flow diagram of an example process for generating data defining a ligand based on the denoised atom state data for each ligand atom.

FIG. 13 is a flow diagram of an example process for jointly training the embedding neural network and the generative model of the design system.

FIG. 14 is a flow diagram of an example process for jointly training an embedding neural network and a generative diffusion model on a training example.

Like reference numbers and designations in the various drawings indicate like elements. Aspects and elements of the figures can be combined. For example, whilst for convenience some implementations and embodiments are described with reference to particular figures, features of implementations and embodiments described with reference to different figures may be combined in a ligand design system.

### DETAILED DESCRIPTION

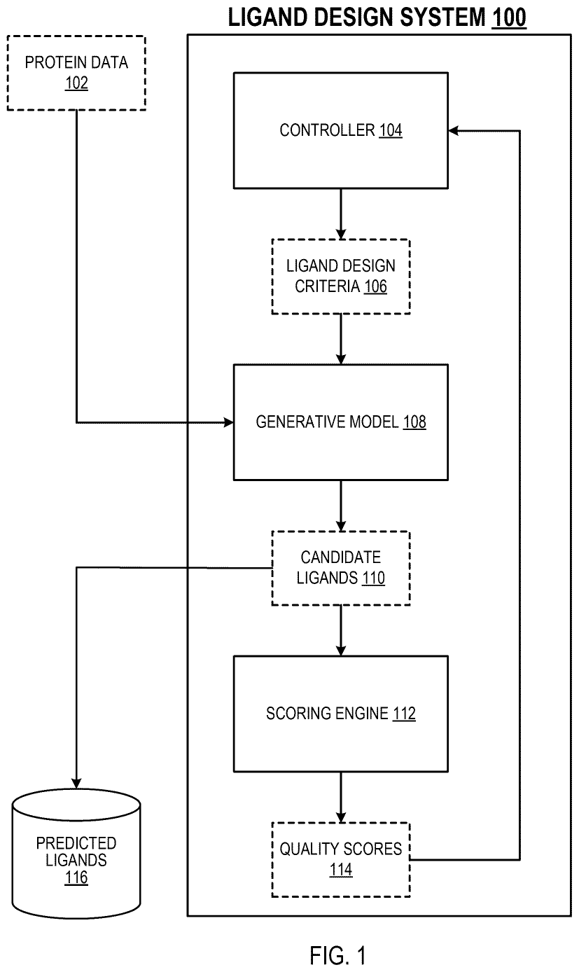

FIG. 1 shows an example ligand design system 100. The ligand design system 100 is an example of a system implemented as computer programs on one or more computers in one or more locations in which the systems, components, and techniques described below are implemented.

The system 100 is configured to computationally design one or more ligands for binding to a protein. More specifically, the system 100 processes an input including protein data 102 characterizing at least a portion of a protein to generate an output that includes a set of predicted ligands 116 that are each predicted to bind to the protein.

The protein data 102 can include any appropriate data characterizing the protein, e.g., data defining one or more amino acid sequences of the protein, or data defining an MSA for the protein, or data characterizing a respective structure of each of one or more template proteins, or a combination thereof. In some cases, the protein data 102 can characterize a full protein or protein complex. In other cases, the protein data 102 can include data characterizing only a portion of a protein or protein complex, e.g., only a binding pocket of a protein.

Each ligand in the set of predicted ligands 116 that are generated by the system 100 can be, e.g., a small molecule, or a protein, or a DNA molecule, or an RNA molecule, and so forth. Each ligand in the set of predicted ligands 116 can be defined by any appropriate data. For instance, each ligand can be defined by data that includes, for each atom in the ligand, respective atom state data for the atom that defines at least a respective 3D spatial position of the atom (e.g., in a complex that includes the protein). The atom state data for an atom can further include any other appropriate atom-specific properties of the atom, such as an elemental type of the atom, a hybridization state of the atom, a partial charge of the atom, and so forth. As another example, a ligand that is a protein can be defined by data that specifies one or more amino acid sequences of the protein. As another example, a ligand that is an RNA molecule or a DNA molecule can be defined by data that specifies one or more nucleic acid sequences of the molecule.

The system 100 operates over a sequence of iterations, referred to for convenience herein as “design iterations.” The system 100 generates a respective set of candidate ligands at each design iteration, and some or all of those candidate ligands can be selected for inclusion in the set of predicted ligands 116 that are output by the system 100, as will be described in more detail below. The set of predicted ligands 116 can include any appropriate number of ligands, e.g., at least 100 ligands, or at least 1000 ligands, or at least 10,000 ligands.

The system 100 can be used to design ligands for any of a variety of applications (downstream tasks). A few example applications of ligands generated by the system 100 are described next.

In one example, the system 100 can be used for drug design, in particular, to generate one or more ligands that are predicted to bind to a target protein that is identified as being involved in a disease process, e.g., associated with cancer, Alzheimer's disease, heart disease, infectious diseases (e.g., bacterial diseases, viral diseases, parasitic diseases, fungal diseases, prion diseases, etc.), and so forth. By binding to the protein, a ligand can modulate (e.g., inhibit or activate) the activity of the protein thereby disrupting the disease process and contributing to treating the disease.

As another example, the system 100 can be used for pest or pathogen control in agriculture, in particular, to generate one or more ligands that are predicted to bind to proteins in agricultural pests or pathogens (e.g., insects, fungi, or bacteria). Such ligands can be used as part of targeted pesticides that bind to proteins that are essential to the survival of pests or pathogens and that are not found in non-target species.

As another example, the system 100 can be used for modifying plant species cultivated for agricultural purposes, in particular, to generate one or more ligands that are predicted to bind to proteins identified as being involved in plant growth, or stress resistance, or both. (Stress resistance in a plant species can characterize an ability of the plant species to survive and adapt to adverse conditions such as drought, high soil salinity, extreme temperatures, and so forth). Such ligands can be used for modulating the behavior of target proteins in the plant species to increase crop yields and stress resistance.

The system 100 can include a controller 104, a generative model 108, and a scoring engine 112, which are each described in more detail next (and throughout this specification).

At each design iteration, the controller 104 generates a current set of ligand design criteria 106 that define desired characteristics of a ligand. The current ligand design criteria 106 can specify: (i) a respective target (desired) value for each of one or more properties of the ligand, or (ii) ligand scaffolding data, or (iii) both.

The target ligand properties can specify a target (desired) value for any appropriate “global” or “atom-specific” properties of the ligand.

Global properties of a ligand can refer to properties that characterize the ligand as a whole rather than being specific to a single atom, and can include properties characterizing one or more of: a binding affinity of the ligand for the protein, absorption properties of the ligand, distribution properties of the ligand, metabolism properties of the ligand, excretion properties of the ligand, toxicity properties of the ligand, a number of rings (e.g., aromatic rings, heterocyclic rings, macrocylic rings) in the ligand, a molecular weight of the ligand, a lipophilicity (log P) of the ligand, an ability of the ligand to donate or accept hydrogen bonds, a total polar surface area of the ligand, a number of rotatable bonds in the ligand, a number of chiral centers (stereocenters) in the ligand, a number of electrophilic (electron-accepting) centers in the ligand, a number of nucleophilic (electron-donating) centers in the ligand, and so forth.

Atom-specific properties of a ligand can refer to properties that relate to specific atoms in the ligand rather than the entire ligand, and can include properties characterizing one or more of: an elemental type of an atom (e.g., carbon, oxygen, nitrogen, and so forth), a hybridization state of an atom (e.g., sp hybridization, or sp² hybridization, or sp³ hybridization, or sp³d hybridization, or sp²d² hybridization, and so forth), a partial charge of an atom, and so forth.

Ligand scaffolding data can specify (at least) a portion of a chemical structure of the ligand, e.g., by specifying at least a portion of a 3D structure of the ligand (e.g., by specifying a respective 3D spatial position of each of one or more atoms in the ligand), or by specifying at least a portion of a molecular sequence (e.g., amino acid sequence or nucleic acid sequence) of the ligand.

Broadly, over the sequence of design iterations, the controller 104 performs an automated, directed search through the space of possible ligand design criteria in order to optimize the “quality scores” of candidate ligands generated in accordance with the ligand design criteria. The quality score of a candidate ligand can characterize the suitability of the candidate ligand for a downstream application, such as for use in a pharmaceutical drug, or for pest or pathogen control, or for modifying plant species cultivated for agricultural purposes, as will be described in more detail below.

At each design iteration after the first design iteration, the controller 104 generates the ligand design criteria for the design iteration in a manner that depends (directly or indirectly) on quality scores of candidate ligands generated at previous design iterations. In particular, the controller 104 can be implemented as any appropriate system that can perform a directed search through the space of possible ligand design criteria over the sequence of design iterations. That is, the search can explore the space of possible ligand design criteria directed (guided) by the quality scores of candidate ligands generated at previous design iterations.

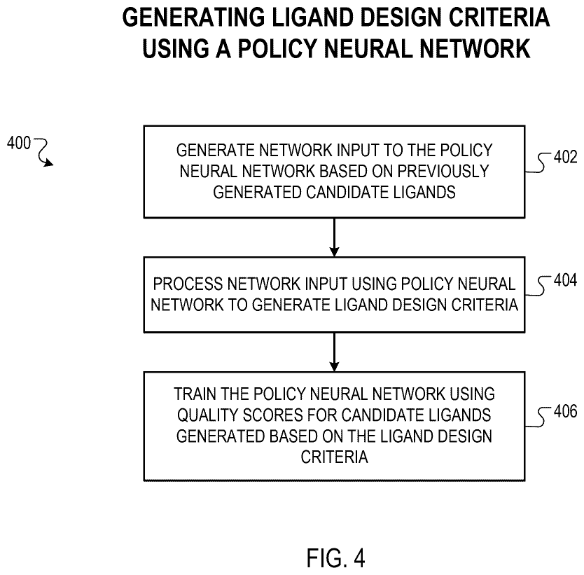

For instance, the controller 104 can be implemented as a neural network, referred to for convenience as a “policy” neural network, that is trained by a reinforcement learning technique. An example process for performing an automated search through the space of possible ligand design criteria using a controller 104 that is implemented as a policy neural network trained by a reinforcement learning technique is described with reference to FIG. 4.

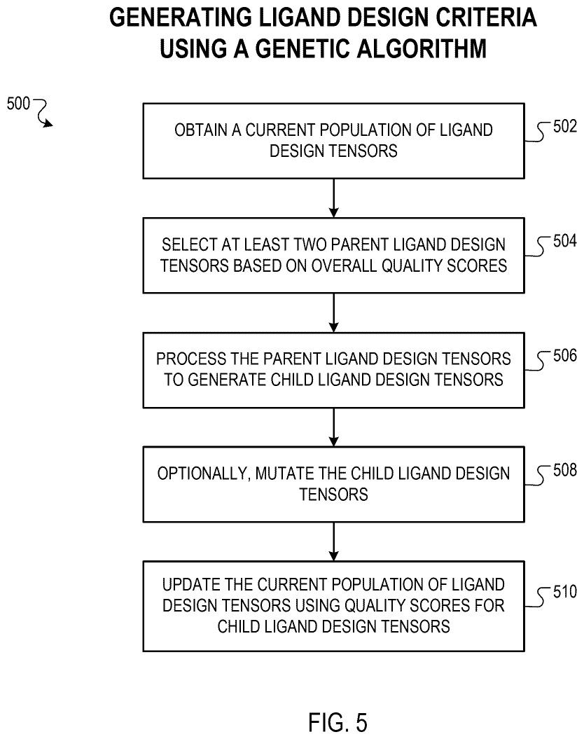

As another example, the controller 104 can be implemented using a genetic algorithm that maintains and iteratively evolves (mutates) a population of ligand design tensors (that each specify a respective set of ligand design criteria). An example process for performing an automated search through the space of possible ligand design criteria using a controller 104 that is implemented using a genetic algorithm is described with reference to FIG. 5. (In embodiments where the controller is implemented using a genetic algorithm, the system can update the controller at each design iteration, e.g., by updating the population of ligand design tensors, as will be described in more detail below).

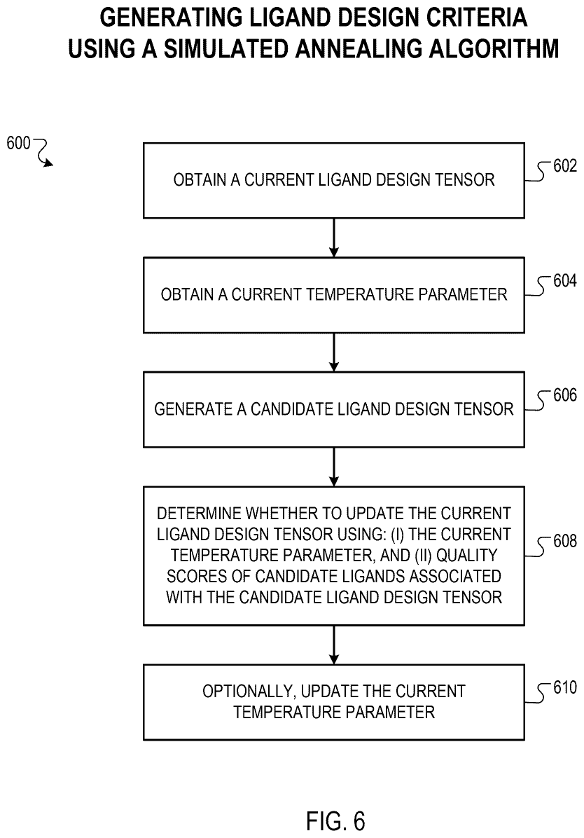

As another example, the controller 104 can be implemented using a simulated annealing algorithm. More specifically, the controller 104 can iteratively propose changes to a ligand design tensor specifying a set of ligand design criteria. The controller 104 can, for example, accept those changes based on a probability that decreases over the sequence of design iterations in a manner that enables the controller to escape local optima by potentially accepting worse ligand design criteria early in the search process. An example process for performing an automated search through the space of possible ligand design criteria using a simulated annealing algorithm is described with reference to FIG. 6.

At each design iteration, the system 100 processes: (i) the protein data 102 characterizing the protein, and (ii) the current ligand design criteria 106, to generate conditioning data for conditioning the generative model 108 (i.e., the current set of ligand design criteria 106 generated by the controller 104 at the design iteration). The generative model 108, when conditioned on data representing the protein 102 and the current ligand design criteria 106, is configured to generate data defining candidate ligands 110. In particular, the generative model 108 attempts to generate candidate ligands 110 that bind to the protein 102 and that satisfy the ligand design criteria 106. For instance, if the ligand design criteria 106 include target ligand properties, then the generative model 108 attempts to generate candidate ligands that have the target ligand properties. As another example, if the ligand design criteria 106 include ligand scaffolding data, then the generative model 108 attempts to generate candidate ligands that have the structure defined by the ligand scaffolding data.

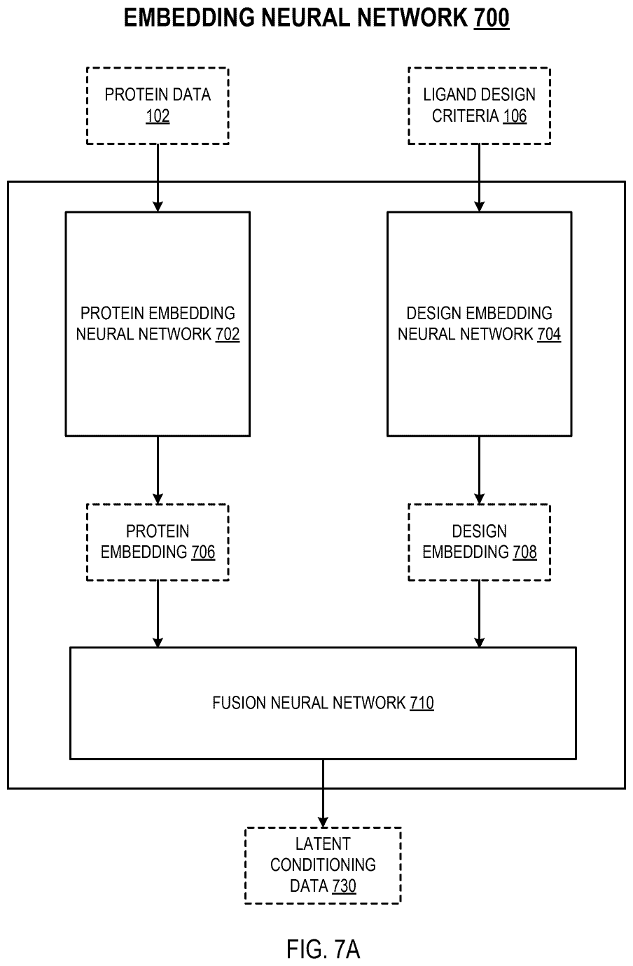

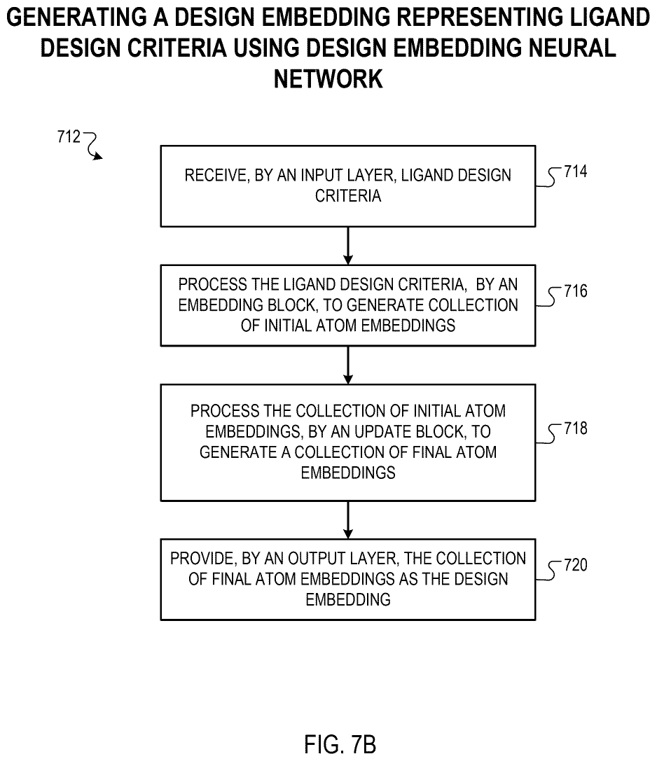

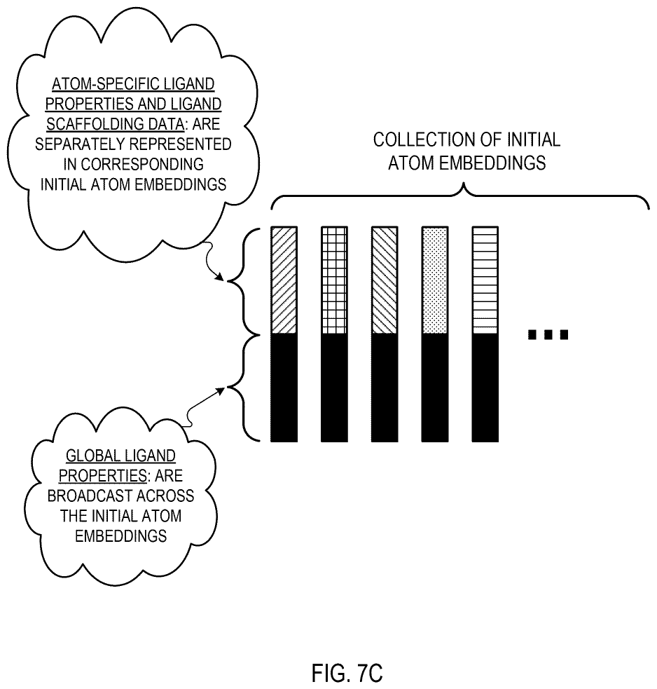

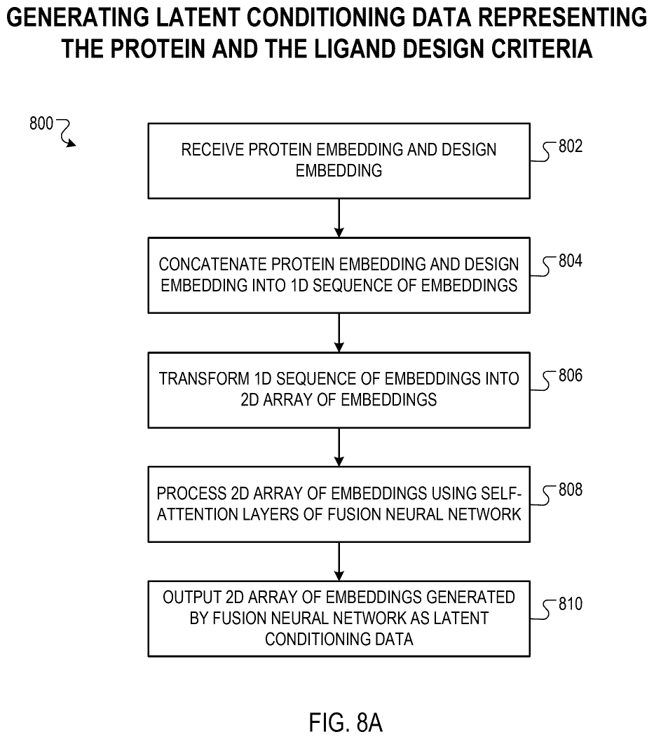

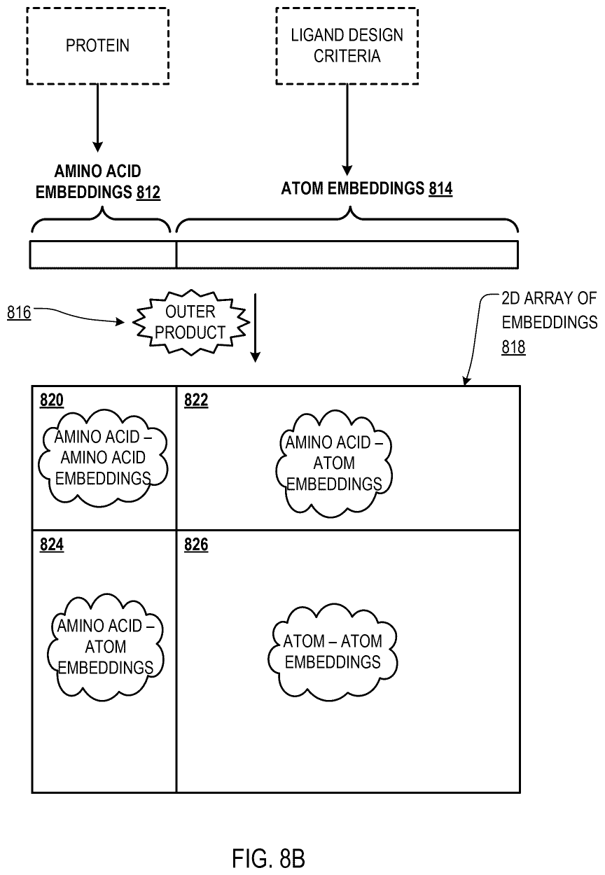

An example process for using an embedding neural network to generate conditioning data for conditioning the generative model based on: (i) the protein data 102 characterizing the protein, and (ii) the ligand design criteria 106 generated by the controller, is described with reference to FIGS. 7A, 7B, 7C, 8A, and 8B. Many other approaches are possible for generating conditioning data representing the current ligand design criteria, e.g., approaches that rely on deterministic and predefined encoding scheme such as one-hot embedding (e.g., instead of using embedding neural networks).

The generative model 108 can be any appropriate conditional generative model. More specifically, the generative model 108 can be any appropriate model that, when conditioned on data representing the protein 102 and the current ligand design criteria 106, can generate samples from a distribution over a space of possible ligands. For instance, the generative model 108 can be implemented as a generative diffusion model, or a generative adversarial neural network (GAN) model (e.g., as described by Goodfellow et al. in “Generative Adversarial Networks,” arXiv:1406.2661), or a flow-based neural network model (normalizing flow model, e.g., such as described by Rezende and Mohamed in “Variational Inference with Normalizing Flows”. Proc. ICML 2015), and so forth.

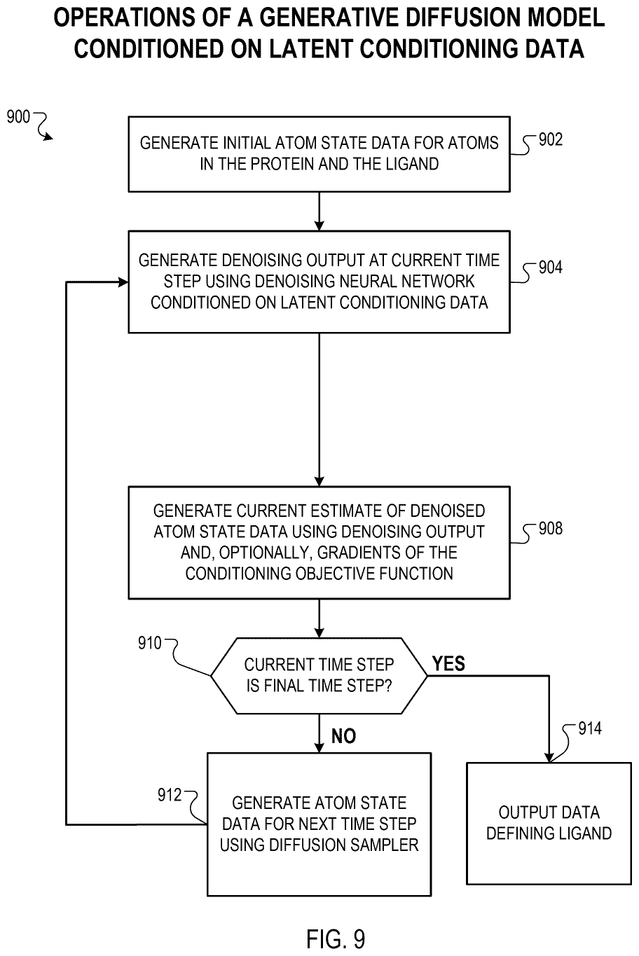

An example process for generating candidate ligands using a generative model 108 that is implemented as a generative diffusion model is described with reference to FIGS. 9, 10, 11, and 12.

In some cases, at some or all of the design iterations, the controller 104 generates multiple distinct sets of ligand design criteria 106 at the design iteration. In these cases, for each set of ligand design criteria 106, the system 100 can generate samples (i.e., defining candidate ligands) from the generative model 108 while the generative model 108 is conditioned on the set of ligand design criteria 106.

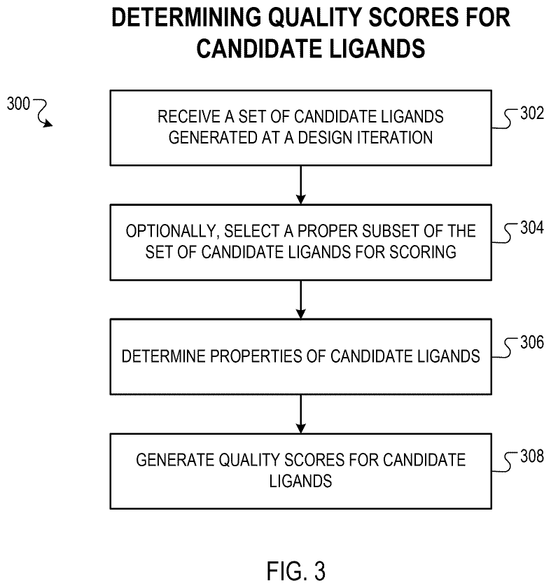

At each design iteration, the scoring engine 112 determines a respective quality score 114 for some or all of the candidate ligands 110 generated at the design iteration. For example, the scoring engine 112 can determine a quality score 114 for each candidate ligand 110 based on predicted properties of the candidate ligand 110. The quality score 114 for a candidate ligand characterizes the suitability of the candidate ligand for a task such as use in a pharmaceutical drug, or pest or pathogen control, or modifying plant species cultivated for agricultural purposes. Example techniques for determining a quality score 114 for a candidate ligand are described below with reference to FIG. 3.

At each design iteration, the system 100 updates the controller 104 using the quality scores 114 for the candidate ligands 110 generated by the generative model 108 at the design iteration. Broadly, the system 100 updates the controller 104 to encourage the generation of candidate ligands 110 with improved (e.g., higher) quality scores 114 at subsequent design iterations. That is, the system 100 updates the controller 104 to generate, at subsequent design iterations, ligand design criteria 106 that, when used to condition the generative model 108, cause the generative model 108 to generate data defining sets of candidate ligands 110 having improved quality scores 114. For example, the system 100 can update values of parameters or other data used by the controller 104 to generate the ligand design criteria to favor generating ligand design criteria 106 that lead to data defining sets of candidate ligands 110 with improved quality scores 114 being generated by the generative model 108 at subsequent design iterations (e.g., the next design iteration). The operations that the system 100 performs to update the controller 104 depend on the implementation of the controller 104. For instance, for a controller 104 implemented as a policy neural network, the system 100 can update the controller 104 by training the policy neural network, e.g., using a reinforcement learning training technique, as will be described in more detail with reference to FIG. 4. As another example, for a controller 104 implemented using a genetic algorithm, the system 100 can update the controller 104 by updating a population of ligand design tensors being maintained and evolved by the genetic algorithm, as will be described in more detail with reference to FIG. 5. As another example, for a controller 104 implemented using a simulated annealing algorithm, the system 100 can update the controller 104 by determining whether to accept changes to a current ligand design tensor being optimized by the controller 104, as will be described in more detail with reference to FIG. 6.

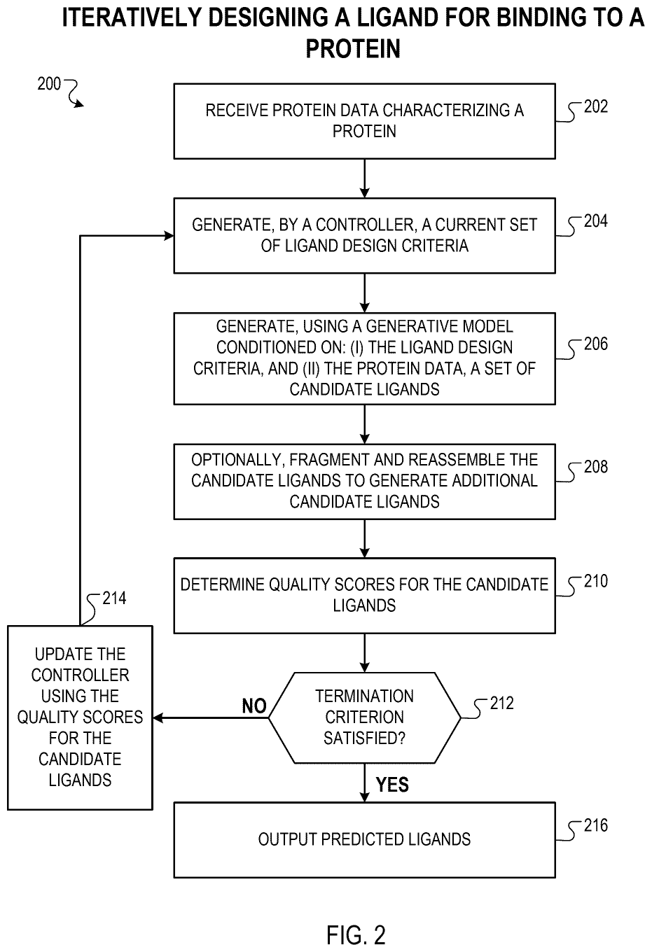

The system can continue performing design iterations until one or more termination criteria are satisfied. The termination criteria can be based on, e.g., a maximum number of design iterations, a maximum compute budget, a maximum time budget, and so forth. After determining that one or more termination criteria are satisfied, the system selects a subset of the candidate ligands generated over the sequence of design iterations for output, as described in more detail below with reference to FIG. 2.

FIG. 2 is a flow diagram of an example process 200 for iteratively designing a ligand for binding to a protein. For convenience, the process 200 will be described as being performed by a system of one or more computers located in one or more locations. For example, a ligand design system, e.g., the ligand design system 100 of FIG. 1, appropriately programmed in accordance with this specification, can perform the process 200.

The system receives protein data characterizing at least a portion of a protein, e.g., data defining one or more amino acid sequences of the protein, or data defining an MSA for the protein, or data characterizing a respective structure of each of one or more template proteins, or a combination thereof (202). The system can receive the protein data from any appropriate source. For instance, the system can receive the protein data, e.g., from a user or an upstream system, e.g., by way of a user interface or an application programming interface (API) made available by the system. In particular, in some cases, the protein data can be transmitted to the system from a remote source over a data communications network, e.g., the internet.

The system performs the steps 204-214 of the process 200 over a sequence of design iterations. For ease of description, the steps 204-214 of the process 200 will be described in the following with reference to a “current” design iteration, which can be any design iteration in the sequence of design iterations.

The system generates, by a controller, a current set of ligand design criteria that define desired characteristics of a ligand (204). The current ligand design criteria 106 can specify: (i) a respective target (desired) value for each of one or more properties of a ligand, or (ii) scaffolding data, or (iii) both. The target ligand properties can specify a target (desired) value for any appropriate “global” or “atom-specific” properties of the ligand. Ligand scaffolding data can, for example, specify (at least) a portion of the 3D structure of the ligand, in particular, by specifying a respective 3D spatial position of each of one or more atoms in the ligand. Specific examples of ligand design criteria are described in more detail above with reference to FIG. 1.

For some or all of the design iterations after the first design iteration, the controller generates the current ligand design criteria in a manner that is based (either directly or indirectly) on the quality scores of candidate ligands generated using ligand design criteria from previous design iterations. More specifically, over the sequence of design iterations, the controller performs an automated, directed search through the space of possible ligand design criteria in order to optimize the quality scores of candidate ligands generated in accordance with the ligand design criteria. Thus, for some or all of the design iterations after the first design iteration, the controller leverages information about the quality scores of candidate ligands generated at previous design iterations in order to inform the generation of the ligand design criteria at the current design iteration.

In particular examples, the controller can be implemented using a policy neural network (as described with reference to FIG. 4), or using a genetic algorithm (as described with reference to FIG. 5), or using a simulated annealing algorithm (as described with reference to FIG. 6). It will be appreciated that these are specific example implementations and that, more generally, the controller can be implemented by any appropriate operations that enable an automated, directed search of and optimization over the space of possible ligand design criteria.

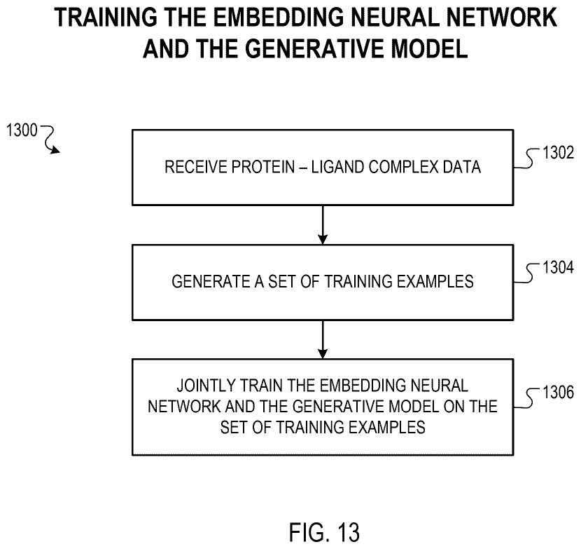

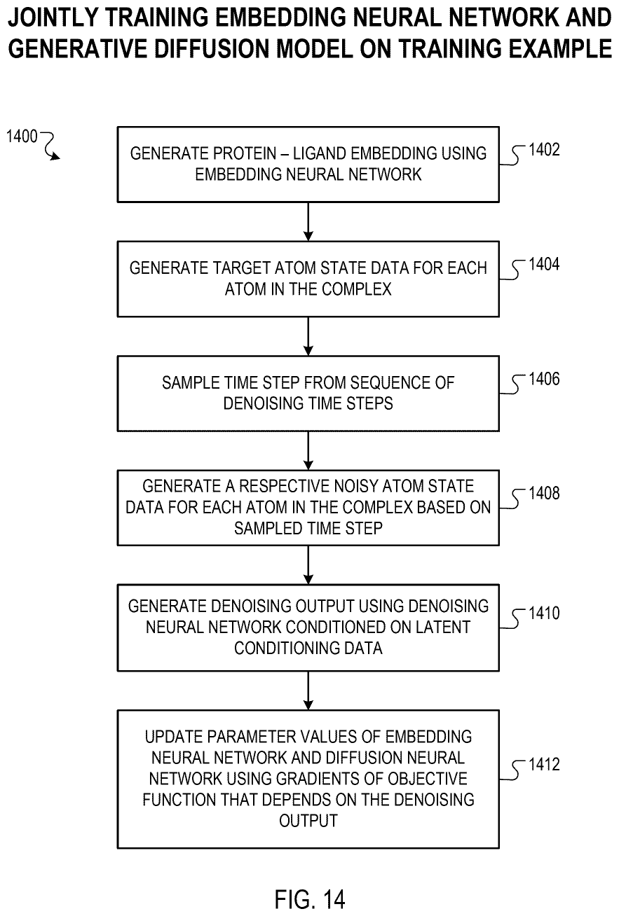

The system generates, using a generative model and while the generative model is conditioned on: (i) the protein data characterizing the protein, and (ii) the ligand design criteria generated by the controller, data defining one or more candidate ligands (206). The generative model attempts to generate candidate ligands that bind to the protein and that satisfy the ligand design criteria. In particular, the generative model has a set of generative model parameters that are configured through training (e.g., by a machine learning training technique) to encourage the generation of candidate ligands that satisfy the ligand design criteria. An example process for training a generative model is described in more detail below with reference to FIG. 13 and FIG. 14. As one example, the generative model can comprise a generative neural network that is trained by backpropagating gradients of an objective function through the generative neural network and adjusting trainable parameters of the generative neural network to optimize the objective function. The objective function can be any appropriate objective function for the candidate ligand generation task, e.g., as described below in connection with FIG. 13.

In some cases, a candidate ligand generated by the generative model may satisfy all the ligand design criteria specified by the conditioning data. However, in other cases, a candidate ligand generated by the generative model may satisfy certain ligand design criteria only approximately, or may entirely fail to satisfy certain ligand design criteria. This may occur, for instance, if the ligand design criteria are mutually incompatible (e.g., if there does not exist a ligand that binds to the protein that simultaneously satisfies all the ligand design criteria), or if the generative model parameters have not been trained on a sufficient amount or type of training data to enable the precise generation of candidate ligands that bind to the protein and that satisfies all the ligand design criteria. In particular, conditioning the generative model on the ligand design criteria increases the likelihood, but does not guarantee, that the generative model will generate candidate ligands that satisfy the ligand design criteria.

As part of conditioning the generative model, the system can generate a conditioning embedding of: (i) the protein data characterizing the protein, and (ii) the ligand design criteria generated by the controller. The system can then provide the conditioning embedding for processing by the generative model. An example process for generating a conditioning embedding for conditioning the generative model based on: (i) the protein data characterizing the protein, and (ii) the ligand design criteria generated by the controller, is described with reference to FIGS. 7A, 7B, 7C, 8A, and 8B.

The generative model can be any appropriate conditional generative model that, when conditioned on data representing the protein and the current ligand design criteria, can generate samples from a distribution over a space of possible ligands. An example process for generating candidate ligands using a generative model that is implemented as a generative diffusion model is described with reference to FIGS. 9, 10, 11, and 12.

The system can generate any appropriate number of candidate ligands using the generative model and while the generative model is conditioned on the protein data and the ligand design criteria. For instance, the system can generate at least 100, or at least 1000, or at least 10,000 candidate ligands at each design iteration.

In some cases, the system can continue generating candidate ligands at the current design iteration until a termination criterion is satisfied. A few examples of possible termination criteria are described next.

In one example, a termination criterion can be that at least a threshold number of candidate ligands have been generated at the current design iteration. The threshold number can be, e.g., 100, or 1000, or 10,000 candidate ligands.

As another example, the termination criterion can be that a rate of increase in a measure of diversity of the candidate ligands generated at the current design iteration fails to satisfy a threshold. More specifically, each time the system generates an additional N candidate ligands using the generative model (where N is any positive integer value, e.g., N=100, or 500, or 1000), the system can determine a measure of diversity of the current set of candidate ligands. The system can then determine a difference between: (i) the current measure of diversity of the set of candidate ligands, and (ii) a previous measure of diversity of the set of candidate ligands, e.g., that is determined before the system generated the most recent N candidate ligands. (The measure of diversity of the initial set of candidate ligands, which is empty, can be defined as zero). In response to determining that the difference between the current measure of diversity and the previous measure of diversity is less than a threshold, the system can determine that the termination criterion has been satisfied. In this manner the system can enable more efficient use of computational resources, e.g., by stopping generation of new candidate ligands when the diversity of the set of candidate ligands is (approximately) saturated.

The system can determine a measure of diversity of a set of candidate ligands in any of a variety of possible ways. For instance, the system can generate a respective embedding of each candidate ligand in the set of candidate ligands, e.g., by generating a molecular fingerprint representing the candidate ligand (e.g., a MACCS Key (Molecular ACCess System) fingerprint or a PubChem fingerprint), or by processing data defining the candidate ligand using an encoder neural network that has been trained to perform an auto-encoding task. The system can then determine the measure of diversity of the set of candidate ligands by computing a measure of diversity of the set of embeddings representing the set of candidate ligands. The system can determine the measure of diversity of a set of embedding representing the set of candidate ligands, e.g., by computing the kurtosis of the set of embeddings, or by computing a sum of the pairwise distances (e.g., Euclidean or cosine distances) between the embeddings in the set of embeddings.

Optionally, the system can augment the set of candidate ligands generated by the generative model by fragmenting and reassembling candidate ligands from the set of candidate ligands (208). For example, multiple candidate ligands from the set of candidate ligands can be fragmented (e.g., by cleaving one or more chemical bonds of the candidate ligand) to obtain a set of candidate ligand fragments from which candidate ligand fragments can be sampled and then combined to generate new candidate ligands for inclusion in the set of candidate ligands. For example, the system can algorithmically break down candidate ligands into smaller fragments and recombine these fragments to form new candidate ligands. Initially, the system can identify particular bonds within a candidate ligand to cleave, generating a set of fragments. The system may then characterize these fragments using computational methods to assess their chemical properties and potential for recombination. During the reassembly phase, the system can use predefined computational rules and algorithms to recombine fragments to increase the likelihood of chemical compatibility and stability of the reassembled candidate ligands. Augmenting the set of candidate ligands by fragmentation and reassembly can increase the diversity of the set of candidate ligands, at a relatively low computational cost, and increase the likelihood of identifying high performing candidate ligands.

The system determines a respective quality score for each of a plurality of the candidate ligands (210). The system can determine the quality score for a candidate ligand based on one more properties of the candidate ligand, and the quality score can characterize a suitability of the candidate ligand for a downstream application, such as use in a pharmaceutical drug, or pest or pathogen control, or modifying plant species cultivated for agricultural purposes. An example process for determining quality scores for candidate ligands is described with reference to FIG. 3.

The system determines whether a termination criterion is satisfied (212). A few examples of possible termination criteria are described next. In one example, a termination criterion can be that a predetermined number of design iterations have been performed. In another example, a termination criterion can be that at least a predefined number of candidate ligands with at least a minimum threshold quality score have been generated. In another example, a termination criterion can be that an amount of computational resources (e.g., measured in floating point operations, FLOPs) consumed as of the current design iteration exceeds a maximum compute budget for designing the ligand. In another example, a termination criterion can be that an amount of time consumed to perform the current number of design iterations exceeds a maximum time budget for designing the ligand.

In response to determining that the termination criteria are not satisfied, the system updates the controller using the quality scores determined for the candidate ligands generated at the current design iteration (214). Broadly, the system updates the controller to encourage the selection of ligand design criteria that result in the generation of candidate ligands with improved quality scores at subsequent design iterations. The specific operations performed by the system to update the controller depend on how the controller is implemented. Example techniques for updating the controller when the controller is implemented using a policy neural network are described with reference to FIG. 4. Example techniques for updating the controller when the controller is implemented using a genetic algorithm are described with reference to FIG. 5. Example techniques for updating the controller when the controller is implemented using a simulated annealing algorithm are described with reference to FIG. 6.

In some cases, as part of updating the controller, the system determines an “overall quality score” based on one or more statistics of the quality scores for the candidate ligands, and optionally, based on features of the set of candidate ligands. For instance, the system can determine an overall quality score as a combination (e.g., a linear combination) of one or more of: a measure of central tendency (e.g., mean or median) of the quality scores of the candidate ligands, or a maximum quality score among the quality scores for the candidate ligands, or a measure of dispersion (e.g., variance) of the quality scores for the candidate ligands, or a measure of diversity of the candidate ligands. (Example techniques for determining a measure of diversity of a set of candidate ligands are described with reference to FIG. 2). The system can use the overall quality score to update the controller, as will be described in more detail with reference to FIG. 4, FIG. 5, and FIG. 6. Determining the overall quality score based at least in part on the diversity of the set of candidate ligands generated at the design iteration can encourage the controller to select ligand design criteria that result in more diversity among the generated ligands, which can increase the likelihood of identifying high performing ligands.

In response to determining that a termination criterion is satisfied, the system can select a subset of the candidate ligands generated over the sequence of design iterations for output (216). The system can output the selected ligands in any appropriate way. For instance, the system can store data defining the selected ligands in a memory, or the system can transmit data defining the selected ligands over a data communications network, e.g., the internet, or the system can provide a representation (e.g., a visual representation) of the selected ligands for display to a user.

The system can determine that a candidate ligand should be selected for output if the candidate ligand satisfies each acceptance criterion in a set of one or more acceptance criteria. The set of acceptance criteria can include any appropriate acceptance criteria. A few examples of possible acceptance criteria are described next.

In one example, an acceptance criterion for a ligand can be that the ligand does not contain any structures that are designated as being physically impossible or highly unstable, e.g., a square planar carbon structure.

In another example, an acceptance criterion for a ligand can be that each atom in the ligand is bonded to at least one other atom in the ligand.

In another example, an acceptance criterion for a ligand can be that the value of a molecular property of the ligand satisfies one or more property-specific thresholds. The molecular property of the ligand can be, e.g., any of the global ligand properties or atom-specific ligand properties described earlier. The property-specific threshold for a molecular property can include, e.g., a lower bound on the value of the property, or an upper bound on the value of the property, or both.

The system can set the one or more property-specific thresholds for a ligand property in any appropriate way. For instance, the system can set a property-specific threshold for a molecular property based on a user input received from a user of the system by way of a user interface or an API made available by the system.

As another example, an acceptance criterion for a ligand can be that, when the candidate ligands are ranked based on the value of a particular molecular property, the candidate ligand is included in a predefined number (e.g., 10, or 100, or 1000) or predefined percentage (e.g., 1%, or 5%, or 10%) of highest (or lowest) ranked candidate ligands. For instance, the molecular property can be binding affinity for the protein, and the acceptance criterion can require that a candidate ligand must be among a predefined number or percentage of top-ranked candidate ligands when ranked based on binding affinity for the protein. In some cases, the respective values of a molecular property for each of the candidate ligands can be relative values, e.g., with respect to a value of the molecular property for a particular ligand.

In order to evaluate whether a candidate ligand satisfies an acceptance criterion (as described above), the system can computationally generate a value of a molecular property for the candidate ligand. Certain molecular properties, such as molecular weight, number of rotatable bonds, number of rings, and so forth, may be directly and unambiguously derivable from the chemical structure of the candidate ligand. However, in order to obtain the values of other molecular properties such as binding affinity, toxicity, absorption, and so forth, the system can process data characterizing the candidate ligand (and, optionally, the protein) using a property prediction model that is configured to generate a predicted value of the molecular property.

The system can implement a property prediction model for a molecular property, e.g., as a machine learning model, e.g., a neural network, or a random forest, or a support vector machine, and so forth, having any appropriate machine learning model architecture. The system can train the property prediction model on a set of training examples and using a machine learning training technique, e.g., optimization of an objective function by stochastic gradient descent.

Each training example can correspond to a molecule and can include: (i) a training input characterizing the molecule, and (ii) an actual value of the molecular property, e.g., as determined using an experimental or computational technique, such as a biological assay, quantum chemistry (e.g., density functional theory) computational technique, or a molecular dynamics computational technique. For each training example, the system can train the property prediction model to reduce a discrepancy between: (i) the actual value of the molecular property specified by the training example, and (ii) a predicted value of the molecular property generated by processing the training input specified by the training example using the property prediction model. The discrepancy between an actual value and a predicted value of a molecular property can be measured, e.g., as an absolute error, or a squared error, or in any other appropriate way. The machine learning training technique can be any appropriate technique appropriate for training the type of machine learning model used to implement the property prediction model. For instance, for a property prediction model implemented as a neural network, the design system can train the property prediction model using a stochastic gradient descent training technique.

Filtering the generated candidate ligands to output only those candidate ligands that satisfy the acceptance criteria can have the effect of reducing the number of candidate ligands that are eligible to be output by the system, e.g., by 50%, or 90%, or 99%. (In some implementations, at each design iteration, the system filters the set of candidate ligands generated at that design iteration, e.g., rather than performing the filtering only after the conclusion of the final design iteration).

Optionally, the system can select one or more of the output ligands for further computational or physical validation.

Computational validation of a ligand can include, e.g., performing computational simulations such as quantum mechanics simulations (e.g., electronic structure calculations or molecular orbitals analysis), or molecular mechanics simulations (e.g., molecular dynamics simulations or Monte Carlo simulations), or docking simulations (e.g., protein-ligand docking simulations), and so forth. Computational simulations of a ligand can generate additional data characterizing the behavior, interactions, and properties of the ligand. In some cases, performing a computational simulation of a ligand can be computationally intensive, in particular, can require significant computational resources such as memory and computing power. Performing computational validation of only the output ligands can thereby significantly reduce consumption of computational resources, e.g. as compared to performing computational validation of all the candidate ligands generated by the system over the sequence of design iterations.

Physical validation of a ligand can include, e.g., physically synthesizing the ligand and (in some cases) experimentally measuring one or more characteristics of the ligand, e.g., by measuring the binding affinity of the ligand for the protein, or by administering a drug including the ligand to a subject (e.g., a cell, or a collection of cells, or an animal, or a person) to assess the absorption, or distribution, or metabolism, or excretion, or toxicity (ADMET) properties of the drug that includes the ligand. The measuring may include, e.g., bringing the candidate ligand into contact with the protein molecule and measuring a change in expression or activity of the protein molecule. Performing physical validation of a ligand can require significant resources, e.g., laboratory resources, chemical resources, personnel resources, and so forth. Performing physical validation of only the output ligands can thereby significantly reduce consumption of resources, e.g., as compared to performing physical validation of all the candidate ligands generated by the system over the sequence of design iterations. Physical validation of a ligand can include, for example, validation of a drug that includes one or more of the candidate ligands, synthesized using any appropriate drug synthesis technique. In general references herein to synthesis can refer to manual and/or automatic synthesis, e.g. using robotic techniques. For example, the drug can be tested for biological activity in vitro or in vivo, e.g., for a therapeutic effect.

FIG. 3 is a flow diagram of an example process 300 for determining quality scores for a set of candidate ligands generated at a current design iteration. For convenience, the process 300 will be described as being performed by a system of one or more computers located in one or more locations. For example, a ligand design system, e.g., the ligand design system 100 of FIG. 1, appropriately programmed in accordance with this specification, can perform the process 300.

The system receives data identifying a set of candidate ligands that are generated at a design iteration, e.g., as described with reference to step 204-214 of FIG. 2 (302).

Optionally, the system selects a proper subset (i.e., fewer than all) of the set of candidate ligands for scoring (304). For instance, the system can determine a partition of the set of candidate ligands into a plurality of clusters (i.e., groups that each include multiple candidate ligands), e.g., using a clustering algorithm (e.g., a k-means or expectation-maximization (EM) clustering algorithm) applied to a set of embeddings representing the set of candidate ligands. The number of clusters may be substantially less (e.g., by one or more orders of magnitude) than the number of candidate ligands in the set of candidate ligands. After performing the clustering, the system can identify a representative candidate ligand for each cluster, and then determine respective quality scores for only the representative candidate ligands. The system can determine a representative candidate ligand for a cluster, e.g., as the candidate ligand that is represented by an embedding that defines a centroid of a group of embeddings representing the candidate ligands in the cluster.

Generating quality scores for fewer than all of the candidate ligands generated at the design iteration, e.g., by clustering the candidate ligands and generating quality scores for only candidate ligands representing respective clusters (as described above), can significantly reduce consumption of computational resources, e.g., memory and computing power. More specifically, generating a quality score for a candidate ligand can require generating one or more computational predictions for properties of the candidate ligand, e.g., using machine learning models, molecular dynamics (MD) simulations, quantum chemistry methods (e.g., density functional theory, Hartree-Fock (HF) methods), molecular mechanics (MM) methods (e.g., force field calculations), and so forth. Generating computational predictions for a candidate ligand can require significant computational resources. Therefore reducing the number of candidate ligands for which quality scores are generated can reduce overall consumption of computational resources.

For each candidate ligand that is selected for scoring, the system determines one or more properties of the candidate ligand (306). For instance, the system can determine properties including one or more of: a binding affinity of the candidate ligand for the protein, absorption properties of the candidate ligand, distribution properties of the candidate ligand, metabolism properties of the candidate ligand, excretion properties of the candidate ligand, toxicity properties of the candidate ligand, a number of rings (e.g., aromatic rings) in the candidate ligand, a molecular weight of the candidate ligand, a lipophilicity (log P) of the candidate ligand, an ability of the candidate ligand to donate or accept hydrogen bonds, a total polar surface area of the candidate ligand, a number of rotatable bonds in the candidate ligand, a number of chiral centers (stereocenters) in the candidate ligand, a number of electrophilic (electron-accepting) centers in the candidate ligand, a number of nucleophilic (electron-donating) centers in the candidate ligand, and so forth.

The system can determine properties of a candidate ligand in any of a variety of possible ways. A few examples techniques for determining properties of a candidate ligand are described next.

In one example, the system can determine certain properties of a candidate ligand by direct, analytic computation, such as molecular weight, number of atoms, number of rings, and so forth.

As another example, the system can determine certain properties of a candidate ligand using a property prediction machine learning model that is configured to process a model input that includes data characterizing the candidate ligand to generate a model output that includes one or more predicted properties of the candidate ligand. The property prediction machine learning model can be any appropriate type of machine learning model, e.g., a neural network, or a random forest, or a decision tree, or a support vector machine, or a linear regression model, and so forth. The property prediction machine learning model can be trained, by a machine learning training technique, on a set of training examples that each include: (i) a training input that includes data characterizing a training molecule, and (ii) a target output that defines one or more properties of the training molecule. In particular, the property prediction machine learning model can be trained to optimize an objective function that, for each training example, measures a discrepancy between: (i) the target output specified by the training example, and (ii) a predicted output generated by the property prediction machine learning model by processing the training input of the training example.

As another example, the system can determine certain properties of a candidate ligand by performing computational chemistry calculations, e.g., including one or more of: molecular dynamics (MD) simulations, quantum chemistry calculations (e.g., density functional theory, Hartree-Fock (HF) methods), molecular mechanics (MM) calculations (e.g., force field calculations), and so forth.

In some implementations, the generative model that generates candidate ligands is configured to generate, for each candidate ligand, an output that defines a three-dimensional (3D) structure of a complex that includes the candidate ligand and the protein, e.g., where the candidate ligand is bound to a binding site on the protein. In these implementations, the system can generate certain properties of each candidate ligand by processing data characterizing the 3D structure of the complex that includes the candidate ligand and the protein (e.g., as opposed to processing only data characterizing the candidate ligand). For instance, to predict the binding affinity of the candidate ligand and the protein, the system can process a model input that is based on the 3D structure of the protein-ligand complex using a property prediction machine learning model to generate a predicted binding affinity score. As another example, to predict the binding affinity of the candidate ligand and the protein, the system can perform molecular dynamics (MD) simulations based on the 3D structure of the protein-ligand complex to generate a predicted binding affinity score.

An example implementation of a generative model that, when conditioned on data characterizing a protein and a set of ligand design criteria, can generate an output that defines a 3D structure of a protein-ligand complex that includes a candidate ligand and the protein is described with reference to FIGS. 9, 10, 11, and 12.

In some cases, the system can distribute the computations to determine predicted properties of the candidate ligands across a group of computing units. The group of computing units can include any appropriate number of computing units, e.g., at least 10, or at least 100, or at least 1000 computing units. The computing units can operate independently and in parallel to generate the predicted properties of the candidate ligands in the set of candidate ligands.

In some cases, certain computing units may be particularly well-suited to performing computations necessary to generate particular predicted properties of candidate ligands. For instance, the set of computing units may include GPUs that are particularly efficient at implementing neural network operations of property prediction neural networks. As another example, the set of computing units may include ASICs that are specially configured for efficiency in performing computational chemistry calculations. Optionally, the system can distribute property prediction tasks across the group of computing units so that particular tasks (e.g., predicting a molecule property using a property prediction neural network, or predicting a molecule property using a computational chemistry calculation) are assigned to corresponding computing units that are identified as being particularly efficient for performing that task.

For each candidate ligand that is selected for scoring, the system generates a quality score for the candidate ligand based on the properties of the candidate ligand (308). For instance, the system can determine, for each of one or more molecule properties, a property-specific score based at least in part on the value of the molecule property for the candidate ligand. The system can then generate the quality score for the candidate ligand by combining (e.g., summing or multiplying) the property-specific scores for each of the molecule properties.

For each molecule property, the system can determine the property-specific score for the molecule property for a candidate ligand by evaluating a scoring function on the value of the molecule property for the candidate ligand. The scoring function can be any appropriate function that maps possible values of the molecule property onto an associated property-specific score value. The scoring function for a molecule property can be designed to assign higher scores to values of the molecule property that indicate a better suitability of the candidate ligand for a downstream task, and lower scores to values of the molecule property that indicate a worse suitability of the candidate ligand for the downstream task. In a particular example, the molecule property may be toxicity and the downstream task may be use of the candidate ligand in a pharmaceutical drug. In this example, the scoring function may be designed to assign a first score (e.g., 100) to toxicity values within a range that is safe, and progressively lower scores to toxicity values that deviate from the safe range.

The scoring functions for the molecule properties can be provided to the system, e.g., by a user of the system. For instance, a user may define scoring functions for various molecule properties by way of a user interface (e.g., a graphical user interface (GUI)) or an application programming interface (API) made available by the system.

FIG. 4 is a flow diagram of an example process 400 for generating ligand design criteria using a controller implemented as a policy neural network. For convenience, the process 400 will be described as being performed by a system of one or more computers located in one or more locations. For example, a ligand design system, e.g., the ligand design system 100 of FIG. 1, appropriately programmed in accordance with this specification, can perform the process 400.

The system generates a network input to the policy neural network (402).

If the current design iteration is the first design iteration, the system can generate a default network input to the policy neural network, e.g., a network input represented as an array of default values, e.g., an array of zeros.

If the current design iteration is after the first design iteration, the system can generate a network input that includes data representing each of one or more candidate ligands generated at one or more previous design iterations, and optionally, a respective quality score for each of the one or more candidate ligands. In some cases, the generative model that generates candidate ligands is configured to generate, for each candidate ligand, an output that defines a three-dimensional (3D) structure of a complex that includes the candidate ligand and the protein, e.g., where the candidate ligand is bound to a binding site on the protein. In these cases, the system can generate a network input that includes, for each of one or more candidate ligands generated at one or more previous design iterations, data representing the structure of a complex that includes the candidate ligand and the protein.

The system can select which previously generated candidate ligands to include in the network input to the policy neural network in any of a variety of possible ways. A few example techniques for selecting previously generated candidate ligands for inclusion in the network input to the policy neural network are described next.

In one example, the system can select a predefined number of candidate ligands that have the highest (e.g., where higher is better) quality scores from among the candidate ligands generated at one or more previous design iterations.

In another example, the system can determine a partition of a set of candidate ligands generated at one or more previous design iterations into a plurality of clusters (i.e., groups that each include multiple candidate ligands), e.g., using a clustering algorithm (e.g., a k-means or expectation-maximization (EM) clustering algorithm) applied to a set of embeddings representing the set of candidate ligands. The number of clusters may be substantially less (e.g., by one or more orders of magnitude) than the number of candidate ligands in the set of candidate ligands. After performing the clustering, the system can identify a representative candidate ligand for each cluster for inclusion in the network input to the policy neural network. The system can determine a representative candidate ligand for a cluster, e.g., as the candidate ligand that is represented by an embedding that defines a centroid of a set of embeddings representing the candidate ligands in the cluster.

The system processes the network input using a policy neural network and in accordance with values of a set of neural network parameters of the policy neural network to generate a network output that defines a set of ligand design criteria (404). The network output that defines the set of ligand design criteria can be represented, e.g., as sequence of numerical values, where each position in the sequence is associated with a respective ligand property (e.g., global ligand property, or atom-specific ligand property, or ligand scaffolding data) and the numerical value that occupies the position defines the desired value of the ligand property. In some cases, the presence of a predefined “null” numerical value (e.g., −1) in a position in the sequence of numerical values defining the ligand design criteria can define that the corresponding ligand property is unspecified in the ligand design criteria.

In some implementations, the policy neural network can be an autoregressive neural network. More specifically, the policy neural network can be configured to generate a sequence of values defining the ligand design criteria one value at a time, starting from a first position in the sequence of values, so that the value selected at each position depends at least in part on the values selected for any preceding positions. For instance, for each position after the first position in the sequence of values defining the ligand design criteria, the system can process an input that includes: (i) the network input generated at step 402, and (ii) data defining the values selected for each preceding position using the policy neural network to generate a score distribution over a set of possible values for the position. The system can then select the value to occupy the position in accordance with the score distribution over the set of possible values for the position. For instance, the system can select the value having the highest score under the score distribution as the value to occupy the position. As another example, the system can process the score distribution (e.g., using a soft-max function) to generate a probability distribution over the set of possible values for the position, and then select the value to occupy the position by stochastically sampling from the probability distribution.

Optionally, if the policy neural network is implemented as an autoregressive neural network (as designed above), the policy neural network can autoregressively generate a sequence of values defining the ligand design criteria without processing data characterizing any of candidate ligands generated at previous design iterations. In particular, in these implementations, the system is not required to perform step 402, i.e., generating a network input to the policy neural network based on previously generated candidate ligands.

The policy neural network can have any appropriate neural network architecture that enables the policy neural network to perform its described functions, e.g., generating a sequence of numerical values that defines a set of ligand design criteria. In particular, the policy neural network can include any appropriate types of neural network layers (e.g., fully connected layers, attention layers, convolutional layers, recurrent layers, and so forth) in any appropriate number (e.g., 3 layers, or 10 layers, or 50 layer) and connected in any appropriate configuration (e.g., as a directed graph of layers). In a particular example, the policy neural network can have a Transformer architecture, e.g., as described with reference to: Ashish Vaswani et al., “Attention is all you need,” Advances in Neural Information Processing Systems 30 (NIPS 2017).

The system updates the values of the set of neural network parameters of the policy neural network using quality scores for candidate ligands generated by the generative model while the generative model is conditioned on the ligand design criteria (406). For instance, the system can determine an overall quality score based on the quality scores for the candidate ligands generated by the generative model while the generative model is conditioned on the ligand design criteria, e.g., as described at step 214 of the process described with reference to FIG. 2. The system can define the overall quality score for the candidate ligands as a reward, and then train the policy neural network using the reward by a reinforcement learning training technique, e.g., a policy gradient training technique (e.g., based on a REINFORCE technique or a proximal policy optimization (PPO) technique, see e.g., arXiv:1707.06347), or an actor-critic training technique, and any other appropriate reinforcement learning technique. Training the policy neural network using the reinforcement learning technique encourages the policy neural network to generate ligand design criteria that increase a cumulative measure of rewards received over the sequence of design iterations. Because the rewards are based on the quality scores of the candidate ligands generated based on the ligand design criteria, training the policy neural network using the reinforcement learning technique thus encourages or biases the policy neural network to generate ligand design criteria that result in the generation of candidate ligands with better (e.g., higher) quality scores.

In another example implementation, the controller can be parameterized by a set of parameters that define, for each ligand design criterion in a set possible ligand design criteria, a respective probability distribution over possible values of the ligand design criterion. The probability distribution can be a parametric probability distribution (e.g., a Normal distribution) or a non-parametric probability distribution (e.g., a discrete distribution that defines a respective probability value for each value in a discrete set of possible values). For each ligand design criterion, the set of possible values for the ligand design criterion can optionally include a “null” value, which would indicate that the value of the ligand design criterion is unspecified.

At each design iteration, the controller can generate a current set of ligand design criteria by, for each ligand design criterion, selecting a value of the ligand design criterion in accordance with the probability distribution over possible values of the ligand design criterion. For instance, the system can select a value for a ligand design criterion by stochastically sampling the value from the probability distribution over the set of possible values of the ligand design criterion, or by selecting a value that has the highest probability under the probability distribution over the set of possible values of the ligand design criterion.

At each design iteration, the system can update the controller by updating the parameters of the controller that define the probability distributions over the possible values of the ligand design criteria. For instance, the system can determine an overall quality score based on the quality scores for the candidate ligands generated by the generative model while the generative model is conditioned on the ligand design criteria, e.g., as described at step 214 of the process described with reference to FIG. 2. The system can define the overall quality score for the candidate ligands as a reward, and then train the parameters of the controller using the reward by a reinforcement learning training technique, e.g., a policy gradient training technique (e.g., based on a REINFORCE technique or a proximal policy optimization (PPO) technique), or an actor-critic training technique, and any other appropriate reinforcement learning technique.

FIG. 5 is a flow diagram of an example process 500 for generating ligand design criteria using a controller implemented using a genetic algorithm. For convenience, the process 500 will be described as being performed by a system of one or more computers located in one or more locations. For example, a ligand design system, e.g., the ligand design system 100 of FIG. 1, appropriately programmed in accordance with this specification, can perform the process 500.

The system obtains a current population (set) of ligand design tensors (502). Each ligand design tensor defines a respective set of ligand design criteria and can be represented as an ordered collection of numerical values, e.g., a vector or matrix or other tensor of numerical values. Each position in each ligand design tensor is associated with a respective ligand property (e.g., global ligand property, or atom-specific ligand property, or ligand scaffolding data) and the numerical value that occupies the position defines the desired value of the ligand property. In some cases, the presence of a predefined “null” numerical value (e.g., −1) in a position in a ligand design tensor defining a set of ligand design criteria can define that the corresponding ligand property is unspecified by the ligand design criteria.

If the current design iteration is the first design iteration, the system can obtain the current population of ligand design tensors, e.g., by stochastically initializing the population of ligand design tensors. More specifically, the system can initialize each ligand design tensor in any of a variety of possible ways. For instance, the system can stochastically select a respective value for each position in the ligand design tensor in accordance with a probability distribution (e.g., a uniform or Normal distribution) over possible values for the position.

If the current design iteration is after the first design iteration, the system can access the current population of ligand design tensors, e.g., from a memory where the system stored the current population of ligand design tensors at the conclusion of the previous design iteration.

Each ligand design tensor in the population of ligand design tensors can be associated with a respective overall quality score. The overall quality score for a ligand design tensor can be based on the quality scores of candidate ligands generated by the generative model while the generative model is conditioned on the ligand design criteria defined by the ligand design tensor, e.g., as described at step 214 of the process described with reference to FIG. 2.

The current population of ligand design tensors can include any appropriate number of ligand design tensors, e.g., at least 10, or at least 100, or at least 1000 ligand design tensors.

The system selects a set of at least two “parent” ligand design tensors from the current population of ligand design tensors based on the overall quality scores for the ligand design tensors in the current population of ligand design tensors (504). The system can select the parent ligand design tensors, e.g., using roulette wheel selection, or tournament selection, or rank-based selection, or using any other appropriate technique. In some cases, the system selects the parent ligand design tensors so that ligand design tensors associated with better (e.g., higher) overall quality scores have a higher likelihood of being selected.

The system processes the set of parent ligand design tensors to generate one or more “child” ligand design tensors, i.e., which each define a respective current set of ligand design criteria generated at the current design iteration (506). More specifically, the system can generate each child ligand design tensor by combining two or more parent ligand design tensors. The system can combine two or more parent ligand design tensors to generate a child ligand design tensor in any of a variety of possible ways. For instance, for one or more positions in a child ligand design tensor, the system can stochastically select one of the parent ligand design tensors, and set the value of the position in the child ligand design tensor equal to the value of the position in the selected parent ligand design tensor. As another example, for one or more positions in a child ligand design tensor, the system can determine the value of the position in the child ligand design tensor as a combination (e.g., an average or sum) of the values in the position in two or more of the parent ligand design tensors.

In some cases, as an alternative to or in combination with generating child ligand design tensors by combining parent ligand design tensors, the system can generate one or more child ligand design tensors by directly mutating (e.g., stochastically modifying) a respective parent ligand design tensor.

Optionally, the system “mutates” one or more of the child ligand design tensors (508). The system can mutate a child ligand design tensor by applying a stochastic modification to one or more positions in the child ligand design tensor, e.g., by replacing values of one or more positions in the child ligand design tensor with random values sampled from respective probability distributions.

The system updates the current population of ligand design tensors using quality scores for candidate ligands generated by the generative model while the generative model is conditioned on each of the child ligand design tensors (510). For instance, for each child ligand design tensor, the system can determine an overall quality score based on the quality scores for candidate ligands generated by the generative model while the generative model is conditioned on the child ligand design tensor, e.g., as described at step 308 of the process 300 described with reference to FIG. 3. The system can then associate each child ligand design tensor with its corresponding overall quality score, and add the child ligand design tensors to the current population of ligand design tensors. Optionally, the system can also filter the current population of ligand design tensors, e.g., to remove one or more ligand design tensors with the worst (e.g., lowest) overall quality scores.

FIG. 6 is a flow diagram of an example process 600 for generating ligand design criteria using a controller implemented using a simulated annealing algorithm. For convenience, the process 600 will be described as being performed by a system of one or more computers located in one or more locations. For example, a ligand design system, e.g., the ligand design system 100 of FIG. 1, appropriately programmed in accordance with this specification, can perform the process 600.

The system obtains a current ligand design tensor (602). The current ligand design tensor defines a set of ligand design criteria and can be represented as an ordered collection of numerical values, e.g., a vector or matrix or other tensor of numerical values. Each position in the current ligand design tensor is associated with a respective ligand property (e.g., global ligand property, or atom-specific ligand property, or ligand scaffolding data) and the numerical value that occupies the position defines the desired value of the ligand property. In some cases, the presence of a predefined “null” numerical value (e.g., −1) in a position in the current ligand design tensor can define that the corresponding ligand property is unspecified by the ligand design criteria.

If the current design iteration is the first design iteration, the system can obtain the current ligand design tensor, e.g., by stochastically initializing the current ligand design tensor. For instance, the system can stochastically select a respective value for each position in the current ligand design tensor in accordance with a probability distribution (e.g., a uniform or Normal distribution) over possible values for the position.

If the current design iteration is after the first design iteration, the system can access the current ligand design tensor, e.g., from a memory where the system stored the current ligand design tensor at the conclusion of the previous design iteration.

The system obtains a current temperature parameter (604). If the current design iteration is the first design iteration, the system can initialize the temperature parameter to a predefined value. If the current design iteration is after the first design iteration, the system can access the current temperature parameter, e.g., from a memory where the system stored the current temperature parameter at the conclusion of the previous design iteration.

The system generates a candidate ligand design tensor based on the current ligand design tensor, where the candidate ligand design tensor defines a current set of ligand design criteria generated at the current design iteration (606). For instance, the system can generate the candidate ligand design tensor by stochastically modifying the respective value in each of one or more positions in the current ligand design tensor. The system can stochastically modify the value in a position of the current ligand design tensor, e.g., by sampling a noise value from a probability distribution (e.g., a uniform distribution or a Normal distribution), and then modifying the value in the position of the current ligand design tensor using the noise value. For instance, the system can add the noise value to the value in the position of the current ligand design tensor, or multiply the noise value with the value in the position of the current ligand design tensor.

The system determines whether to update the current ligand design tensor using: (i) the current temperature parameter, and (ii) quality scores for candidate ligands generated by the generative model while the generative model is conditioned on the candidate ligand design tensor (608). For instance, the system can determine an overall quality score based on the quality scores of candidate ligands generated by the generative model while the generative model is conditioned on the candidate ligand design tensor, e.g., as described at step 214 of the process described with reference to FIG. 2. If the overall quality score of the candidate ligand design tensor is better (e.g., higher) than an overall quality score of the current ligand design tensor, then the system can set the current ligand design tensor equal to the candidate ligand design tensor. If the overall quality score of the candidate ligand design tensor is worse (e.g., lower) than an overall quality score of the current ligand design tensor, then the system can set the current ligand design tensor equal to the candidate ligand design tensor with a probability that is based at least in part on the current temperature parameter. For instance, the system can determine the probability P as:

$$P = exp ( - \frac{Q ( {D}^{′} ) - Q ( D )}{T} ) \qquad ( 1 )$$

where Q(D′) is the overall quality score of the candidate ligand design tensor, Q(D) is the overall quality score of the current ligand design tensor, and T is the current temperature parameter.

Optionally, the system updates the current temperature parameter, e.g., by decreasing the value of the current temperature parameter in accordance with a predefined schedule (610). Generally, the simulated annealing algorithm allows exploration of the space of possible ligand design criteria by, in some cases, allowing changes to the current ligand design tensor that result in a worsening of the overall quality score associated with the current ligand design tensor. Over the sequence of design iterations, the system can reduce the value of the temperature parameter and thus reduce the likelihood of transitioning to ligand design tensors associated with worse overall quality scores.

FIG. 7A shows an example embedding neural network 700 that can generate conditioning data for conditioning the generative model that generates candidate ligands, as described with reference to FIG. 1. More specifically, the embedding neural network 700 is configured to process: (i) protein data 102 characterizing a protein, and (ii) ligand design criteria 106 specifying target ligand properties and/or scaffolding data, to generate latent conditioning data 730 representing the protein 102 and the ligand design criteria 106.

The embedding neural network 700 includes a protein embedding neural network 702, a design embedding neural network 04, and a fusion neural network 710, which are each described in more detail next (and throughout this specification).

The protein embedding neural network 702 is configured to process the protein data 102 characterizing the protein to generate a protein embedding 706 of the protein. The protein embedding 706 can include, e.g., a respective amino acid embedding of each amino acid in each amino acid sequence of the protein.

The protein embedding neural network 702 can have any appropriate neural network architecture that enables the protein embedding neural network 702 to perform its described functions. In particular, the protein embedding neural network 702 can include any appropriate types of neural network layers (e.g., fully connected layers, convolutional layers, attention layers, etc.) in any appropriate number (e.g., 5 layers, 10 layers, or 20 layers) and connected in any appropriate configuration (e.g., as a directed graph of layers).

A particular example of a possible architecture of the protein embedding neural network 702 is the “Evoformer” neural network described in Jumper et al., “Highly accurate protein structure prediction with AlphaFold,” Nature, Vol 596, 26 Aug. 2021. The Evoformer can process a network input derived from: (i) the amino acid sequence of a protein, (ii) an MSA for the protein, and (iii) the 3D structures of one or more template amino acid sequences, to generate an output that includes a “single representation” that defines a respective embedding of each position in each amino acid sequence of the protein.

The design embedding neural network 704 is configured to process the target ligand properties and/or ligand scaffolding data specified by the ligand design criteria 106 to generate a design embedding 708.

The design embedding 708 can include a respective atom embedding representing each atom in a set of possible atoms that are eligible for inclusion in the ligand. The number of atoms that are included in the ligand to be generated by the design system may be unknown. Therefore, the number of atom embeddings in the design embedding 708 can be defined by the ligand design criteria 106. Each atom embedding in the design embedding thus represents an atom that may (or may not) be selected for inclusion in the ligand generated by the generative model when conditioned on the latent conditioning data 730, as will be described in more detail below. The number of atom embeddings in the design embedding 708 can thus define a maximum number of atoms that can be included in a ligand generated by the generative model. The atoms in the set of possible atoms that are eligible for inclusion in the ligand may be referred to for convenience as “ligand atoms”.

The design embedding neural network 704 can have any appropriate neural network architecture that enables the design embedding neural network 704 to perform its described functions. In particular, the design embedding neural network 704 can include any appropriate types of neural network layers (e.g., fully connected layers, convolutional layers, attention layers, etc.) in any appropriate number (e.g., 5 layers, 10 layers, or 20 layers) and connected in any appropriate configuration (e.g., as a directed graph of layers).

An example of processing ligand design criteria 106 using a design embedding neural network 704 to generate a design embedding 708 is described in more detail with reference to FIG. 7B.

The fusion neural network 710 is configured to process the protein embedding 706 and the design embedding 708 to generate the latent conditioning data 730 that jointly represents the protein 102 and the ligand design criteria 106. The fusion neural network 710 can have any appropriate neural network architecture that enables the fusion neural network 710 to perform its described functions. In particular, the fusion neural network 710 can include any appropriate types of neural network layers (e.g., fully connected layers, convolutional layers, attention layers, etc.) in any appropriate number (e.g., 5 layers, 10 layers, or 20 layers) and connected in any appropriate configuration (e.g., as a directed graph of layers).

An example of processing a protein embedding 706 and a design embedding 708 using a fusion neural network 710 to generate latent conditioning data 730 is described in more detail with reference to FIG. 8A.

FIG. 7B is a flow diagram of an example process 712 for processing ligand design criteria using a design embedding neural network (e.g., that is included in the embedding neural network described with reference to FIG. 7A) to generate a design embedding. For convenience, the process 712 will be described as being performed by a system of one or more computers located in one or more locations. For example, a ligand design system, e.g., the ligand design system 100 of FIG. 1, appropriately programmed in accordance with this specification, can perform the process 712.

The system receives, by an input layer of the design embedding neural network, ligand design criteria specifying one or more target (desired) properties of the ligand and/or ligand scaffolding data (714). The ligand design criteria can specify global properties of the ligand (i.e., that characterize the ligand as a whole rather than being specific to a single atom) or atom-specific properties of the ligand (i.e., that relate to specific atoms in the ligand rather than the entire ligand) or both. The ligand scaffolding data can, for example, specify a respective 3D spatial position for each of one or more ligand atoms.

The system processes the ligand design criteria, by an embedding block of the design embedding neural network, to generate a collection of initial atom embeddings that includes a respective initial atom embedding for each ligand atom (716). If the ligand design criteria specify a maximum number of atoms that can be selected for inclusion in the ligand, then the system can generate a number of initial atom embeddings equal to the maximum number of atoms that can be selected for inclusion in the ligand. If the ligand design criteria do not specify a maximum number of atoms that can be selected for inclusion in the ligand, then the system can generate a default (e.g., predefined) number of initial atom embeddings.

The system can include data representing the global properties of the ligand in all of the initial atom embeddings. For each ligand atom, the system can include: (i) any atom-specific properties of the ligand atom, and (ii) any ligand scaffolding data specifying a 3D spatial position of the atom, in the initial atom embedding of the atom. Thus, for each ligand atom, the system can generate the initial atom embedding for the atom based on “atom property data” that includes: (i) any global properties of the ligand, (ii) any atom-specific properties of the atom, and (iii) any ligand scaffolding data for the atom.

To generate the initial atom embedding for an atom, the system can generate an array (e.g., vector) of numerical values having a predefined dimensionality, where each component of the array is assigned to represent a respective type of atom property data, e.g., global ligand property data, atom-specific property data, and ligand scaffolding data. For instance, the array can include one or more components assigned to represent a target binding affinity of the ligand for the protein, and one or more components assigned to represent an elemental type of the atom, and one or more components assigned to represent ligand scaffold data for the atom, and so forth. The system can populate the array with atom property data for the atom that includes: (i) any global properties of the ligand, (ii) any atom-specific properties of the atom, and (iii) any ligand scaffolding data for the atom. Any components of the array that are not populated using the atom property data are masked, e.g., are set to a default value (e.g., negative one).

In some implementations, the array of atom property data for an atom directly defines the initial atom embedding of the atom. In other implementations, the system processes the array of atom property data for an atom using one or more neural network layers (e.g., fully connected layers) of the embedding block to generate the initial atom embedding for the atom.

For one or more of the ligand atoms, the array of atom property data for the atom may be partially or fully masked, e.g., if the ligand design criteria do not specify any global properties of the ligand, any atom-specific properties of the atom, or any ligand scaffolding data for the atom.

The system processes the collection of initial atom embeddings, by an update block of the design embedding neural network, to generate a collection of final atom embeddings that includes a respective final atom embedding for each ligand atom (718). In some implementations, the update block includes a sequence of one or more self-attention neural network layers that are each configured to receive a collection of atom embeddings, apply one or self-attention operations (e.g., query-key-value (QKV) self-attention operations) to the collection of input atom embeddings to update each of the atom embeddings, and then output the collection of updated atom embeddings. The first self-attention layer in the sequence of self-attention layers can receive as input the collection of initial atom embeddings generated by the embedding block, and each subsequent self-attention layer can receive as input the collection of atom embeddings output by the preceding self-attention layer. The final self-attention layer in the sequence of self-attention layers can output collection of final atom embeddings.

The system provides the collection of final atom embeddings, by an output layer of the design embedding neural network, as the design embedding representing the ligand design criteria (720).

FIG. 7C provides an illustration of a collection of initial atom embeddings generated by an embedding block of a design embedding neural network. Each initial atom embedding represents a respective ligand atom. Any global ligand properties specified by the ligand design criteria are broadcast across all of the initial atom embeddings. Any atom-specific properties or ligand scaffolding data that are specific to a particular atom are separately represented in the initial atom embedding for that atom.

FIG. 8A is a flow diagram of an example process 800 for processing a protein embedding of a protein and a design embedding of ligand design criteria using a fusion neural network to generate latent conditioning data that jointly represents the protein and the ligand design criteria. For convenience, the process 800 will be described as being performed by a system of one or more computers located in one or more locations. For example, a ligand design system, e.g., the ligand design system 100 of FIG. 1, appropriately programmed in accordance with this specification, can perform the process 800.

The system receives the protein embedding of the protein and the design embedding of the ligand design criteria (802). The protein embedding can be generated by a protein embedding neural network, as described with reference to FIG. 3A, and can include a respective amino acid embedding for each position in each amino acid sequence of the protein. The design embedding can be generated by a design embedding neural network, as described with reference to FIG. 3B, and can include a respective atom embedding for each ligand atom in a set of ligand atoms.

The system concatenates the protein embedding and the design embedding to generate a one-dimensional (1D) sequence of embeddings (804). The 1D sequence of embedding includes the amino acid embeddings of the protein embedding and the atom embeddings of the design embedding. The embeddings included in the 1D sequence of embeddings can be ordered in any appropriate way, e.g., the 1D sequence of embeddings can be ordered to have the amino acid embeddings followed by the atom embeddings, or to have the atom embeddings followed by the amino acid embeddings. The length of the 1D sequence of embeddings can be a sum of: (i) the number of amino acid embeddings in the protein embedding, and (ii) the number of atom embeddings in the design embedding. The 1D sequence of embeddings can be represented by data having dimensionality NumTokens×d, where NumTokens is given by the sum of: (i) the number of amino acid embeddings in the protein embedding, and (ii) the number of atom embeddings in the design embedding, and d is a positive integer value defining the number of channel dimensions in each amino acid embedding and atom embedding.

The system processes the 1D sequence of amino acid embeddings and atom embeddings to generate data defining a two-dimensional (2D) array of embeddings (806). The 2D array of embeddings can be represented by data having dimensionality NumTokens×NumTokens×d′ where (as above) NumTokens is given by the sum of: (i) the number of amino acid embeddings in the protein embedding, and (ii) the number of atom embeddings in the design embedding, and d′ is a positive integer value defining the number of channel dimensions in each embedding (d′ can, for example, be equal to d, i.e., the number of channel dimensions in each amino acid embedding and atom embedding). The system can generate the 2D array of embeddings from the 1D sequence of embeddings in any of a variety of ways. For instance, the system can generate the 2D array of embeddings as a result of an element-wise outer product of the 1D sequence of embeddings with itself. As another example, the system can generate the 2D array by an appropriate 2D concatenation operation, e.g., where the embedding at each position (i, j) in the 2D array of embeddings is generated by concatenating: (i) the embedding at position i, and (ii) the embedding at position j, in the 1D sequence of amino acid embeddings (where indices i, j∈{1, . . . N}, where N is the length of the 1D sequence of embeddings).

Each embedding in the 2D array of embeddings can be, e.g.: (i) an atom-atom embedding, or (ii) an amino acid-amino acid embedding, or (iii) an amino acid-atom embedding. Each atom-atom embedding is derived from a pair of atom embeddings representing ligand atoms. Each amino acid-amino acid embedding is derived from a pair of amino acid embeddings representing amino acids in the protein. Each amino acid-atom embedding is derived from a pair of embeddings that includes an amino acid embedding representing an amino acid in the protein and an atom embedding representing a ligand atom.

The system processes the 2D array of embeddings by a set of neural network layers of the fusion neural network to generate an updated 2D array of embeddings defining the latent conditioning data (808). The updated 2D array of embeddings can have the same dimensionality as the original 2D array of embeddings, e.g., NumTokens×NumTokens×d′ (where NumTokens and d′ are defined as above). To generate the updated 2D array of embeddings, the fusion neural network can process the 2D array of embeddings using a sequence of one or more self-attention blocks. Each self-attention block can be configured to receive the current 2D array of embeddings as an input, to update the current 2D array of embeddings by one or more self-attention operations (e.g., single-head or multi-head query-key-value (QKV) self-attention operations), and to provide the updated 2D array of embeddings to a subsequent neural network layer (e.g., to another self-attention block, or to an output layer of the fusion neural network).

The self-attention blocks of the fusion neural network can implement any appropriate self-attention operations. A few examples of self-attention operations that can be implemented by self-attention blocks of the fusion neural network are described next.

In some implementations, one or more of the self-attention blocks of the fusion neural network implement “row-wise” or “column-wise” self-attention over the current 2D array of embeddings (i.e., that is provided as an input to the self-attention block). In a row-wise self-attention operation, a self-attention layer updates each given embedding in the 2D array of embeddings using a self-attention operation over only embeddings located in the same row as the given embedding in the 2D array of embeddings. In a column-wise self-attention operation, a self-attention block updates each given embedding in the 2D array of embeddings using a self-attention operation over only embeddings located in the same column as the given embedding in the 2D array of embeddings.

In some implementations, one or more of the self-attention blocks of the fusion neural network implement triangle self-attention operations. An example implementation of triangle self-attention operations are described in Jumper et al., “Highly accurate protein structure prediction with AlphaFold,” Nature, Vol 596, 26 Aug. 2021. A triangle self-attention operation can be one that performs an attention operation on a triplet of embeddings, in particular between one embedding and a pair of other embeddings, more particularly using a triangle concept to provide consistency of respective relationships between the embedding and each of the other embeddings.

In some implementations, one or more of the self-attention blocks of the fusion neural network implement a full self-attention operation over the current 2D array of embeddings, e.g., by updating each embedding in the 2D array of embeddings using attention over the entire 2D array of embeddings.

The fusion neural network can include other neural network layers, i.e., in addition to the sequence of self-attention blocks, e.g., other neural network layers (such as fully connected layers or normalization layers) that are interleaved among the self-attention blocks. The fusion neural network can also include features such as skip connections, e.g., to implement residual blocks in the fusion neural network.

The system outputs the 2D array of embeddings generated by the fusion neural network as the latent conditioning data (810). The system can provide the latent conditioning data generated by the fusion neural network, e.g., for conditioning the generative model.

FIG. 8B illustrates operations performed by the ligand design system to generate the latent conditioning data. The system generates a protein embedding that includes a sequence of amino acid embeddings 812 (one for each amino acid in each amino acid sequence of the protein) and a design embedding that includes a sequence of atom embeddings 814 (one for each ligand atom in a set of ligand atoms). The design system concatenates the sequence of amino acid embeddings and the sequence of atom embeddings into a 1D sequence of embeddings, and then transforms the 1D sequence of embeddings (e.g., by an outer product operation) into a 2D array of embeddings. The 2D array of embeddings can include: (i) atom-atom embeddings 826, (ii) amino acid-amino acid embeddings 820, and (iii) amino acid-atom embeddings 822, 824. Each atom-atom embedding is derived from a pair of atom embeddings representing ligand atoms. Each amino acid-amino acid embedding is derived from a pair of amino acid embeddings representing amino acids in the protein. Each amino acid-atom embedding is derived from a pair of embeddings that includes an amino acid embedding representing an amino acid in the protein and an atom embedding representing a ligand atom. The ligand design system can process the 2D array of embeddings 818 using a sequence of one or more self-attention blocks (e.g., that implement row-wise attention, or column-wise attention, or triangle self-attention, or full self-attention) to generate the latent conditioning data.

FIG. 9 is a flow diagram of an example process 900 for generating data defining a ligand that is predicted to bind to a protein using a generative diffusion model that includes a denoising neural network. For convenience, the process 900 will be described as being performed by a system of one or more computers located in one or more locations. For example, a ligand design system, e.g., the ligand design system 100 of FIG. 1, appropriately programmed in accordance with this specification, can perform the process 900. A similar process can be used with other generative models that are similar to a generative diffusion model, e.g., a consistency model.

The system generates respective initial atom state data for each atom in the protein and for each ligand atom in the ligand (902).

The atom state data for an atom can include features that can be categorized as “continuous features” or “categorical features.”

A continuous feature refers to a feature that can take on values from a continuous range of possible values, e.g., in a continuous interval. The atom state data for an atom can include continuous features such as a feature defining a 3D spatial position of the atom (e.g., which can assume values from in the continuous space [symbol] ³), a feature defining a partial charge of the atom (e.g., which can assume values in the continuous interval [−1,+1]), and so forth.

A categorical feature can refer to a feature that can assume a value in a finite set of possible values. The atom state data for an atom can include categorical features such as a feature defining an elemental type of the atom (e.g., which can assume values from a discrete set of possible elemental types, e.g., carbon, oxygen, nitrogen, etc.), a feature defining a hybridization state of the atom (e.g., which can assume values from a discrete set of possible hybridization states, e.g., sp hybridization, or sp² hybridization, or sp³ hybridization, or sp³d hybridization, or sp²d² hybridization, etc.), and so forth.

Generally, for each atom, the atom state data for the atom specifies at least a 3D spatial position of the atom. Optionally, the atom state data for each atom can further specify one or more additional atom-specific properties of the atom, e.g., including one or more of: an elemental type of the atom, a hybridization state of the atom, a partial charge of the atom, and so forth.

The system can stochastically sample the initial atom state data for some or all of the atoms in the complex, i.e., for some or all of the protein atoms in the protein and for some or all of the ligand atoms in the ligand.

In particular, for some or all of the continuous features in the atom state data for an atom, the system can sample a value of the feature from a probability distribution over the space of possible values of the feature. The system can then include the sampled value of the feature in the initial atom state data for the atom.

For instance, the system can sample a 3D spatial position of an atom from a probability distribution over 3D space, and then include the sampled 3D spatial position in the initial atom state data for the atom. The probability distribution over 3D space can be, e.g., a standard Normal distribution over 3D space. The system can represent the sampled 3D spatial position in the initial atom state data for the atom, e.g., in Cartesian coordinates or in spherical coordinates or in any other appropriate coordinate system over 3D space.

As another example, the system can sample a value for a partial charge of an atom from a probability distribution (e.g., a uniform distribution) over a range of possible partial charges (e.g., the interval [−1,+1]). The system can then include the sampled value of the partial charge in the initial atom state data for the atom.

Further, for some or all of the categorical features in the atom state data for an atom, the system can sample a distribution over a set of possible values of the feature. The system can then include the distribution over possible values of the feature in the initial atom state data for the atom. The distribution over the set of possible values of the feature can define a respective score for each value in the set of possible values of the feature. The system can sample the score for each possible value of the feature from a probability distribution over a range of possible scores. Optionally, the system can normalize the distribution over the set of possible values of the feature prior to including the distribution in the initial atom state data, e.g., by processing the distribution using a soft-max function.

For instance, the system can generate a distribution over a set of possible atomic element types of an atom, e.g., by sampling a respective score for each possible atomic element type from a probability distribution over a range of possible scores, e.g., a uniform distribution over the range [0,1]. The system can then include the distribution over the set of possible atomic element types of the atom in the initial atom state data for the atom.

As another example, the system can generate a distribution over a set of possible atomic hybridization states of an atom, e.g., by sampling a respective score for each possible atomic hybridization state from a probability distribution over a range of possible scores, e.g., a uniform distribution over the range [0,1]. The system can then include the distribution over the set of possible atomic hybridization states of the atom in the initial atom state data for the atom.

In some cases, the system receives scaffolding data (e.g., ligand scaffolding data) that specifies a respective target 3D spatial position of each of one or more atoms in the ligand. If the system receives scaffolding data specifying a target 3D spatial position of an atom in the ligand, then the system can generate initial atom state data for that atom that includes the target 3D spatial position of the atom rather than stochastically sampled 3D spatial position data for the atom (as described above).

In some cases, the system receives ligand design criteria that specify one or more atom-specific target properties of ligand atoms in the ligand (e.g., target hybridization state data, or target partial charge data). If the system receives ligand design criteria specifying an atom-specific target property of a ligand atom, then the system can generate initial atom state data for that atom that includes the atom-specific target property of the ligand atom rather than stochastically sampled atom property data (as described above).

In particular, if the system receives ligand design criteria specifying a particular value of a continuous feature of an atom (e.g., a 3D spatial position or a partial charge of the atom), then the system can include that feature value in the initial atom state data for the atom (e.g., instead of a stochastically sampled value for that feature).

If the system receives ligand design criteria specifying a particular value of a categorical feature of an atom (e.g., an element type of the atom or a hybridization state of the atom), then the system can generate a distribution over a set of possible values of the feature that assigns a first value (e.g., one) to the actual value of the feature and a second value (e.g., zero) to each other value of the feature. For instance, the system can generate a one-hot distribution over the set of possible values of the feature that uniquely identifies the actual value of the feature. The system can then include the generated distribution over possible values of the feature in the initial atom state data for the atom (e.g., instead of a stochastically sampled distribution over possible values of the feature).

For each atom in the complex, the initial atom state data for the atom thus defines a “noisy” representation of the state of the atom. The system performs steps 904-912, which are described next, over a sequence of iterations that may be referred to as “denoising time steps,” to progressively denoise the initial atom state data for the atoms as part of designing a ligand that binds to the protein. More specifically, the system progressively denoises the initial atom state data for each atom in the complex to cause the noisy representation of the atom state to converge on a denoised representation that accurately represents the atom state.

The description of steps 904-910 which follows will reference a “current” denoising time step for convenience; the current denoising time step can be any denoising time step in the sequence of denoising time steps. The system can perform the steps 904-910 over any appropriate number of denoising time steps, e.g., 3 denoising time steps, 10 denoising time steps, or 100 denoising time steps. The number of denoising time steps can be a predetermined number of time steps.

The system generates a denoising output by processing respective current atom state data for the atoms in the complex using a denoising neural network that is conditioned on the latent conditioning data (904). If the current denoising time step is the first denoising time step, then the current atom state data for the atoms in the complex may be the initial atom state data, e.g., as generated at step 902. If the current denoising time step is after the first denoising time step, then the current atom state data for the atoms in the complex may be generated at the preceding denoising time step, e.g., as in step 912 of the process 900, which will be described in more detail below.

The denoising output can be any appropriate data that enables estimation of the denoised (ground truth) atom state data of each atom in the complex. For instance, the denoising output can define, for each atom in the complex, a predicted error in the atom state data of the atom at the current denoising time step. As another example, the denoising output can directly define, for each atom in the complex, predicted atom state data for the atom. As another example, the denoising output can define, for each atom in the complex, both: (i) a predicted error in the atom state data for the atom at the current denoising time step, and (ii) predicted atom state data for the atom. As another example, the denoising output can define, for each atom in the complex, a prediction for an array of values that is a linear combination of: (i) actual atom state data for the atom, and (ii) an error between the atom state data for the atom at the current denoising time step and the actual atom state data for the atom, e.g., as implemented by the v-parametrization described in: Tim Salimans, Jonathan Ho, “Progressive distillation for fast sampling of diffusion models,” ICLR 2022, arXiv:2202.00512v2.

The system generates a current estimate of the denoised atom state data for each atom in the complex using the denoising output generated by the denoising neural network (908). The system can generate the current estimate of the denoised atom state data for each atom in the complex in any appropriate way, depending on the form of the denoising output. A few example techniques for generating the current estimate of the denoised atom state data for each atom in the complex using the denoising output are described next.

In one example, the denoising output defines, for each atom, a respective prediction for the atom state data of the atom. In this example, the respective predicted atom state data for each atom defines the current estimate of the denoised atom state data for the atom.

In another example, the denoising output defines, for each atom, a predicted error in the atom state data for the atom at the current time step. In this example, the system can generate the current estimate for the denoised atom state data for each atom as a linear combination of: (i) the current atom state data for the atom, and (ii) the predicted error in the atom state data for the atom. Each term in the linear combination can be scaled by a respective constant value that is dependent on the time step. For instance, the system can generate the current estimate for the denoised atom state data xₜ₋₁ for an atom in the complex as:

$${x}_{t - 1} = {α}_{t}^{0.5} ( {x}_{t} - ( 1 - {α}_{t} ) {( 1 - {\overline{α}}_{t} )}^{- 0.5} {ϵ}_{θ} ( {x}_{t} , t ) ) \qquad ( 2 )$$

where t indexes the current time step, αₜ, α̅ₜ, and σₜ are constants specific to time step t, and ϵ_{θ}(xₜ, t) is the predicted error in the atom state data of the atom (e.g., as generated by the denoising neural network at the time step). (In the notation of equation (2), the time steps decrement, such that time step t−1 is the “next” time step after time step t). The constants in equation (2) (αₜ and α̅ₜ) can be selected in accordance with a predefined noise schedule. For example αₜ=1−βₜ, α̅ₜ can be the cumulative product of values of αₜ from an initial time step up to current time step t, and βₜ (which represents a noise schedule of the forward process) can decrease, e.g. from 1 to 0 (e.g. linearly or with a cosine or any other dependence), as the time steps decrement. In the example of equation (2) the sampling is deterministic and the noise variance, σₜ² =0 (in a probabilistic setting it can depend on 1−αₜ and a mean μₜ₋₁, rather than xₜ₋₁, can be determined).

In another example, the denoising output defines, for each atom, both: (i) predicted atom state data for the atom, and (ii) a predicted error in the atom state data for the atom at the current time step. In this example, the system can generate the current estimate for the denoised atom state data for the atom as a combination (e.g., an average) of: (i) the predicted atom state data for the atom as specified by the denoising output, and (ii) predicted atom state data for the atom that is derived from the predicted error in the atom state data for the atom at the current time step, e.g., using equation (2).

In another example, the denoising output is expressed using a v-parametrization, and the system generates a respective current estimate for the denoised atom state data for each atom using the techniques described in Tim Salimans, Jonathan Ho, “Progressive distillation for fast sampling of diffusion models,” ICLR 2022, arXiv:2202.00512v2.

If the current denoising time step is not the final denoising time step, the system generates respective atom state data for each atom in the complex for the next denoising time step based on the current estimates of the denoised atom state data for the atoms (as generated at step 908) using an appropriate diffusion sampling technique (912). A few examples of possible diffusion sampling techniques are described next.

In one example, the system can generate the atom state data for each atom in the complex at the next denoising time step by combining random noise with the current estimate of the denoised atom state data for the atom. For instance, for each atom, the system can add respective random noise to the current estimate of the denoised atom state data for the atom. The random noise can be sampled from an appropriate probability distribution. The probability distribution can vary based on the time step, e.g., such that the variance of the noise combined with the updated atom state data for the atoms decreases over the sequence of time steps. An example is the denoising diffusion probabilistic model (DDPM), e.g., as described in Ho et al. arXiv:2006:11239.

As another example, the system can generate the atom state data for each atom in the complex at the next denoising time step using a deterministic diffusion sampling technique, i.e., that does not rely on random noise. An example of a deterministic diffusion sampling technique is the denoising diffusion implicit model (DDIM), e.g., as described in: Jiaming Song, Chenlin Meng, Stefano Ermon, “Denoising diffusion implicit models,” ICLR 2021, arXiv:2010.02502v4.

Optionally, the system can refrain from applying the diffusion sampling technique to any component of the atom state data for an atom that was initialized at step 902 to represent target values specified by the ligand design criteria. More specifically, for any component of the atom state data for an atom that was initialized at step 902 to represent a target value specified by the ligand design criteria, the system can set the value of the component in the atom state data for the atom for the next denoising time step to be the same as the value of the component in the current estimate of the denoised atom state data.

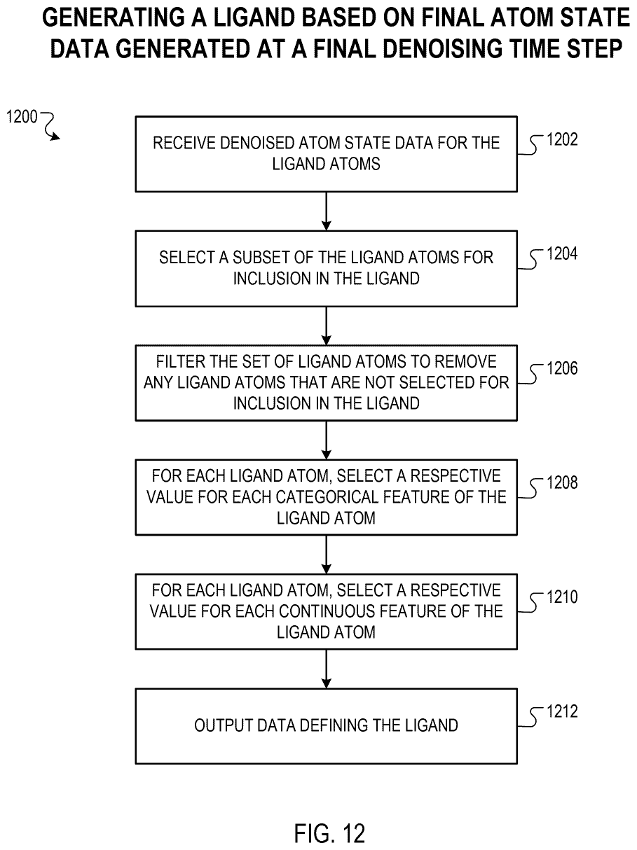

If the current denoising time step is the final denoising time step in the sequence of denoising time steps, the system generates data defining the ligand based on the denoised atom state data for the ligand atoms (914). The denoised atom state data for an atom refers to the current estimate of the denoised atom state data generated for the atom at the final denoising time step in the sequence of denoising time steps. An example process for generating data defining the ligand based on the denoised atom state data is described with reference to FIG. 12.

Optionally, the system can additionally output data specifying a 3D structure of the protein, i.e., as defined by the 3D spatial position data included in the denoised atom state data for each atom in the protein.

Optionally, the system can additionally output data specifying a 3D structure of the entire protein-ligand complex, i.e., as defined by the 3D spatial position data included in the denoised atom state data for each atom in the protein and for each atom in the ligand.

Optionally, the system can additionally output data identifying the binding pocket where the ligand is bound to the protein. In particular, the system can identify any amino acid residue in the protein which includes an atom that is within a threshold distance (e.g., 2 Angstroms) of at least one atom in the ligand as being included in the binding pocket. The data identifying the binding pocket can include data identifying each of the amino acid residues in the protein that are included in the binding pocket.

Optionally, the system can perform the process 900 multiple times to generate multiple ligands for binding to the protein. Each execution of the steps of the process 900 can result in the generation of a different ligand, e.g., as a result of stochasticity in the random sampling performed to generate the initial atom state data for the atoms (at step 902), and in some cases, as a result of stochasticity in the diffusion sampler (at step 912).

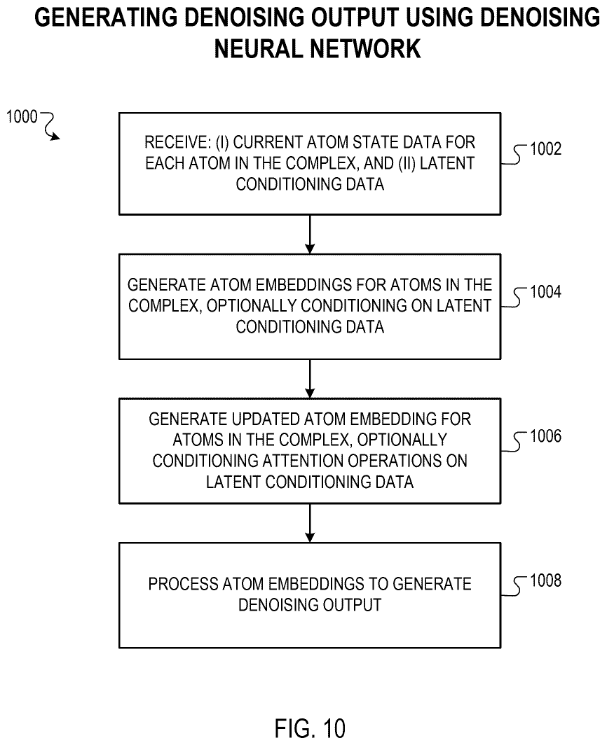

FIG. 10 is a flow diagram of an example process 1000 for generating a denoising output using a denoising neural network conditioned on latent conditioning data. For convenience, the process 1000 will be described as being performed by a system of one or more computers located in one or more locations. For example, a design system, e.g., the design system 100 of FIG. 1, appropriately programmed in accordance with this specification, can perform the process 1000.

The system receives: (i) data defining respective current atom state data for each atom in the complex, and (ii) the latent conditioning data (1002). The latent conditioning data can include: (i) atom-atom embeddings, (ii) amino acid-amino acid embeddings, and (iii) amino acid-atom embeddings. The system can optionally receive additional inputs, e.g., an input defining a current denoising time step in a diffusion process being implemented by a generative diffusion model.

The system generates a respective atom embedding for each atom in the complex, using an encoder block of the generative neural network, based at least in part on the current atom state data for the atom (1004). Optionally, the system can generate the atom embeddings for the atoms based at least in part on the latent conditioning data (and, optionally, the current time step in the diffusion process), e.g., in addition to the current atom state data for the atoms. For instance, for each atom, the system can generate the atom embedding of the atom based on both: (i) the current atom state data for the atom, and (ii) a respective conditioning embedding selected from the collection of embeddings included in the latent conditioning data. For an atom included in an amino acid in the complex, the conditioning embedding for the atom can be the amino acid-amino acid embedding (i.e., from the latent conditioning data) of the amino acid that includes the atom (i.e., the amino acid-amino acid embedding corresponding to a pair of amino acids that includes two copies of the amino acid). For a ligand atom, the conditioning embedding for the atom can be the atom-atom embedding (i.e., from the latent conditioning data) of the atom (i.e., the atom-atom embedding corresponding to a pair of atoms that includes two copies of the atom).

The system can generate the atom embedding for an atom based on the current atom state data for the atom (and, optionally, a conditioning embedding for the atom) in any of a variety of possible ways. For instance, the system can generate the atom embedding for an atom by processing the atom state data for the atom using an encoder block of the denoising neural network. As another example, the system can generate the atom embedding for an atom by processing both: (i) the atom state data for the atom, and (ii) the conditioning embedding for the atom, using an encoder subnetwork of the denoising neural network. As another example, the system can generate the atom embedding for an atom by concatenating: (i) an output generated by an encoder subnetwork of the denoising neural network by processing the atom state data for the atom, and (ii) the conditioning embedding for the atom.

Optionally, for each atom, the system can further generate the respective atom embedding of the atom based on data, e.g., a set of binary flags, indicating which (if any) components of the initial atom state data are initially set to target values specified by the ligand design criteria, e.g., as opposed to being stochastically sampled.

The encoder block of the denoising neural network can include any appropriate types of neural network layers (e.g., fully connected layers, convolutional layers, and so forth) in any appropriate number (e.g., 1 layer, 3, layers or 5 layers) and connected in any appropriate configuration (e.g., as a directed graph of layers). In a particular example, the encoder block includes a sequence of fully connected neural network layers and is configured to, for each atom, process data defining the atom state data for the atom and the conditioning embedding for the atom to generate the atom embedding of the atom.

In some implementations, for each amino acid in the protein, the system can generate one atom embedding that jointly represents all the atoms in the amino acid. Thus, in these implementations, the number of atom embeddings may be equal to a sum of: (i) the number of ligand atoms, and (ii) the number of amino acids in the protein. The system can generate an atom embedding that jointly represents all the atoms in an amino acid in any appropriate way. For instance, the system can generate a respective atom embedding for each atom in the amino acid (as described above), and then combine the atom embeddings for the atoms in the amino acid, e.g., using a pooling operation (e.g., a max pooling or a summation pooling operation), or by processing the atom embeddings for the atoms in the amino acid using one or more neural network layers (e.g., fully connected layers or self-attention layers) to generate the embedding that jointly represents all the atoms in the amino acid. Generating atom embeddings that jointly represent all the atoms in an amino acid can significantly reduce the overall number of atom embeddings and thus reduce consumption of computational resources, e.g., memory and computing power, resulting from operations performed by an update block of the denoising neural network, as will be described next.

The system processes the atom embeddings for the atoms in the complex, using an update block of the denoising neural network, to generate a respective updated atom embedding for each atom in the complex (1006). The update block of the denoising neural network can include a sequence of self-attention blocks. Each self-attention block can be configured to receive a respective current atom embedding for each atom in the complex, to apply a self-attention operation to the current atom embeddings of the atoms in the complex, and to provide the updated atom embeddings, e.g., for processing by a subsequent neural network layer.

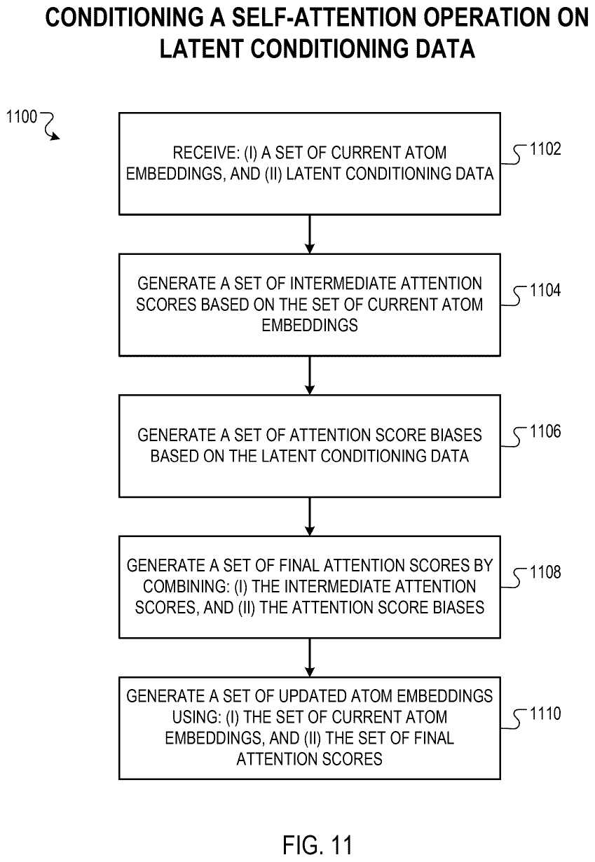

Each self-attention block included in the update block can apply any appropriate self-attention operation to the current atom embeddings of the atoms in the complex, e.g., a single-head or multi-head query-key-value (QKV) self-attention operation. Optionally, the system can condition the self-attention operations of one or more of the self-attention blocks on the latent conditioning data. An example process for implementing a self-attention operation conditioned on the latent conditioning data is described in more detail with reference to FIG. 11.

The update block of the denoising neural network can include any appropriate number of self-attention blocks (e.g., 1 self-attention block, or 10 self-attention blocks, or 50 self-attention blocks) and can optionally include additional neural network layers of any appropriate type (e.g., fully connected layers, convolutional layers, and so forth) in any appropriate number (e.g., 1 layer, 3, layers or 5 layers) and connected in any appropriate configuration (e.g., interleaved with the self-attention blocks).

The system processes the updated atom embeddings (i.e., as generated by the update block of the denoising neural network) using a decoder block of the denoising neural network to generate the denoising output (1008). The decoder block of the denoising neural network can include any appropriate types of neural network layers (e.g., fully connected layers, convolutional layers, and so forth) in any appropriate number (e.g., 1 layer, 3, layers or 5 layers) and connected in any appropriate configuration (e.g., as a directed graph of layers).

In a particular example, the decoder block can include a sequence of fully connected neural network layers that are configured to operate separately on each updated atom embedding to generate a predicted error in the atom state data of the corresponding atom at the current time step, or to generate predicted atom state data of the corresponding atom, or both.

In implementations where one atom embedding jointly represents all the atoms in an amino acid, the decoder block can process that (updated) atom embedding to generate respective denoising outputs for all the atoms in the amino acid. For instance, the decoder block can process an updated atom embedding that jointly represents all the atoms in an amino acid to generate a respective predicted error in the atom state data of each atom in the amino acid, or to generate respective atom state data of each atom in the amino acid, or both.

FIG. 11 is a flow diagram of an example process 1100 for updating a set of current atom embeddings using a self-attention operation that is implemented by a self-attention block of the denoising neural network and that is conditioned on the latent conditioning data. For convenience, the process 1100 will be described as being performed by a system of one or more computers located in one or more locations. For example, a design system, e.g., the design system 100 of FIG. 1, appropriately programmed in accordance with this specification, can perform the process 1100.

The system receives: (i) a set of current atom embeddings, and (ii) latent conditioning data (1102). The set of current atom embeddings includes a respective atom embedding for each atom in the complex. (In some cases, for each amino acid in the protein, all the atoms in the amino acid are jointly represented by one atom embeddings). The set of current atom embeddings can be generated, e.g., by the encoder block of the denoising neural network or by a previous self-attention layer in the update block of the denoising neural network. The latent conditioning data can be generated by an embedding neural network. The latent conditioning data can include: (i) atom-atom embeddings, (ii) amino acid-amino acid embeddings, and (iii) amino acid-atom embeddings.

The system generates a set of intermediate attention scores based on the set of current atom embeddings (1104). The set of intermediate attention scores includes a respective attention score for each pair of current atom embeddings from the set of current atom embeddings.

The system can generate the intermediate attention scores in any of a variety of possible ways. For instance, the system can generate a respective query embedding for each current atom embedding by processing the current atom embedding using a query neural network, e.g., as:

$$Q = {W}^{Q} \cdot E \qquad ( 3 )$$

where Q is a matrix where each column (or row) defines a respective query embedding, W^{Q} is a matrix of parameter values (defining the query neural network in this example), and E is a matrix where each column (or row) defines a respective current atom embedding. Further, the system can generate a respective key embedding for each current atom embedding by processing the current atom embedding using a key neural network, e.g., as:

$$K = {W}^{K} \cdot E \qquad ( 4 )$$

where K is a matrix where each column (or row) defines a respective key embedding, W^{K} is a matrix of parameter values (defining the key neural network in this example), and E is a matrix where each column (or row) defines a respective current atom embedding. The system can generate the intermediate attention scores based on the query embeddings and the key embeddings, e.g., as:

$$A = Q \cdot {K}^{T} \qquad ( 5 )$$

where A is a matrix of intermediate attention scores, Q is the matrix of query embeddings, and K is the matrix of key embeddings.

The system generates a set of attention score biases based on the latent conditioning data (1106). The set of attention score biases includes a respective attention score bias for each pair of current atom embeddings from the set of current atom embeddings.

The system can generate the set of attention score biases in any of a variety of possible ways. For instance, for each pair of current atom embeddings, the system can generate the attention score bias for the pair of current atoms embeddings by processing a corresponding conditioning embedding selected from the collection of embeddings included in the latent conditioning data using a projection neural network.

For a pair of current atom embeddings that includes: (i) a first current atom embedding representing a first ligand atom, and (ii) a second current atom embedding representing a second ligand atom, the conditioning embedding can be the atom-atom embedding for the first ligand atom and the second ligand atom in the latent conditioning data.

For a pair of current atom embeddings that includes: (i) a first current atom embedding representing an atom included in an amino acid, and (ii) a second current atom embedding representing a ligand atom, the conditioning embedding can be the amino acid-atom embedding corresponding to: (i) the amino acid that includes the atom, and (ii) the ligand atom, in the latent conditioning data.

For a pair of current atom embeddings that includes: (i) a first current atom embedding representing an atom included in a first amino acid, and (ii) a second current atom embedding representing an atom included in a second amino acid, the conditioning embedding can be the amino acid-amino acid embedding corresponding to: (i) the first amino acid, and (ii) the second amino acid, in the latent conditioning data.

For a pair of current atom embeddings that includes: (i) a first current atom embedding that jointly represents the atoms in a first amino acid, and (ii) a second current atom embedding that jointly represents the atoms in a second amino acid, the conditioning embedding can be the amino acid-amino acid embedding corresponding to: (i) the first amino acid, and (ii) the second amino acid, in the latent conditioning data.

For a pair of current atom embeddings that includes: (i) a first current atom embedding that jointly represents the atoms in an amino acid, and (ii) a second current atom embedding that represents a ligand atom, the conditioning embedding can be the amino acid-atom embedding corresponding to: (i) the amino acid, and (ii) the ligand atom, in the latent conditioning data.

The projection neural network can have any appropriate neural network architecture that enables the projection neural network to perform its described functions, e.g., processing a conditioning vector to generate an attention score bias. In particular, the projection neural network can include any appropriate number of neural network layers (e.g., 1 layer, or 5 layers, or 10 layers) in any appropriate number (e.g., 1 layer, 3, layers or 5 layers) and connected in any appropriate configuration (e.g., as a directed graph of layers).

The system generates a set of final attention scores by combining: (i) the intermediate attention scores, and (ii) the attention score biases (1108). The set of final attention scores includes a respective final attention score for each pair of current atom embeddings in the set of current atom embeddings. The system can generate the final attention score for a pair of current atom embeddings by combining (e.g., summing): (i) the intermediate attention score for the pair of current atom embeddings, and (ii) the attention score bias for the pair of current atom embeddings. Optionally, the system can apply further processing operations to the set of final attention scores, e.g., by applying a soft-max operation to some or all of the final attention scores.

The system generates a set of updated atom embeddings using: (i) the set of current atom embeddings, and (ii) the set of final attention scores (1110). For instance, to generate the set of updated atom embeddings, the system can generate a respective value embedding for each current atom embedding in the set of current atom embeddings by processing the current atom embedding using a value neural network, e.g., as:

$$V = {W}^{V} \cdot E \qquad ( 6 )$$

where V is a matrix where each column (or row) defines a respective value embedding, W^{V} is a matrix of parameter values (defining the value neural network in this example), and E is a matrix where each column (or row) defines a respective current atom embedding. The system can then generate the set of updated atom embeddings, e.g., as:

$${E}^{′} = V \cdot A \qquad ( 7 )$$

where each column (or row) of E′ defines a respective updated atom embedding, each column (or row) of V defines a respective value embedding, and A denotes the set of final attention scores arranged into a matrix.

In implementations where the self-attention block implements a multi-head attention operation, each head of the attention operation can individually perform the steps of the process 1100, and the updated atom embeddings generated by each attention head can be combined (e.g., concatenated) to define the overall output of the multi-head attention operation. Each attention head can have a respective set of neural network parameters, having values that are specific to each attention head, that are used for generating the intermediate attention scores and the attention score biases.

FIG. 12 is a flow diagram of an example process 1200 for generating data defining a ligand based on the denoised atom state data for each ligand atom. For convenience, the process 1200 will be described as being performed by a system of one or more computers located in one or more locations. For example, a design system, e.g., the design system 100 of FIG. 1, appropriately programmed in accordance with this specification, can perform the process 1200.

The system receives respective denoised atom state data for each ligand atom (1202). The denoised atom state data for a ligand atom refers to a current estimate of the denoised atom state data generated for the ligand atom at the final denoising time step in the sequence of denoising time steps.

The system selects a subset (e.g., proper subset, i.e., one or more but not all) of the set of ligand atoms for inclusion in the ligand (1204). In particular, as described above, each ligand atom in the set of ligand atoms represents an atom that is eligible for inclusion in the ligand. The system is not aware, prior to generating the denoised atom state data for the ligand atoms, which ligand atoms will be selected for inclusion in the ligand and which ligand atoms will be discarded. The number of ligand atoms in the set of ligand atoms thus represents the maximum number of atoms that can be included in the ligand.

For each ligand atom, the system can determine whether to select the ligand atom for inclusion in the ligand based on the 3D spatial position of the ligand atom as defined by the denoised atom state data for the ligand atom. For instance, the system can determine that a ligand atom should be selected for inclusion in the ligand only if the 3D spatial position of the ligand atom is at least a threshold distance from a predefined 3D spatial position referred to as the “throw-away”position. In this example, the system may have trained the generative diffusion model to move ligand atoms that are not included in the ligand to the throw-away position.

The system filters the set of ligand atoms to remove any ligand atoms that are not selected for inclusion in the ligand (1206). Filtering the set of ligand atoms to remove ligand atoms that are not selected for inclusion in the ligand may reduce the number of ligand atoms in the set of the ligand atoms by any appropriate amount, e.g., by 10%, or 50%, or 90%. After the filtering, a ligand atom is included in the set of ligand atoms if and only if it represents an atom that is selected for inclusion in the ligand. In some cases, the system may select all the ligand atoms in the original set of ligand atoms for inclusion in the ligand and thus no ligand atoms are filtered from the set of ligand atoms.

For each ligand atom in the set of ligand atoms, the system selects a respective value for each categorical feature that is represented in the denoised atom state data for the ligand atom (1208). A categorical feature can refer to a feature having a value that is selected from a finite set of possible values. Examples of categorical features can include, e.g., atomic hybridization state or atomic element type. The denoised atom state data for an atom can represent a categorical feature by a distribution over the set of possible values of the categorical feature, where the distribution assigns a respective score to each possible value of the categorical feature.

The system can select a value for a categorical feature of a ligand atom based on the distribution over possible values of the feature that is included in the denoised atom state data for the atom in any of a variety of possible ways. For instance, the system can select the value that is assigned the highest score by the distribution over the set of possible values of the feature as the value of the categorical feature. As another example, the system can sample the value of the categorical feature from a probability distribution defined by the distribution over the set of possible values of the feature.

For each ligand atom in the set of ligand atoms, the system selects a respective value for each continuous feature that is represented in the denoised atom state data for the ligand atom (1210). A continuous feature can refer to a feature having a value that is selected from a continuous set of possible values. Examples of continuous features can include, e.g., 3D spatial position, atomic partial charge, and so forth. The denoised atom state data for an atom can directly represent a continuous feature in one or more assigned dimensions of the denoised atom state data, and the system can select the value for the continuous feature by extracting the corresponding dimensions representing the value of the continuous feature from the denoised atom state data for the atom.

The system provides data defining the ligand (1212). The data defining the ligand includes data identifying each atom that is included in the ligand, and a set of features associated with each atom that is included in the ligand. The set of features associated with an atom that is included in the ligand can include continuous features, e.g., the 3D spatial position of the atom and the partial charge of the atom, and/or categorical features, e.g., the hybridization state of the atom and the element type of the atom.

FIG. 13 is a flow diagram of an example process 1300 for jointly training the embedding neural network and the generative model of the design system. In the example process 1300, the generative model is a model that implements a differentiable generative process. For instance, the generative model can be a generative diffusion model implemented using a denoising neural network. For convenience, the process 1300 will be described as being performed by a system of one or more computers located in one or more locations. For example, a design system, e.g., the design system 100 of FIG. 1, appropriately programmed in accordance with this specification, can perform the process 1300.

The system receives data characterizing a set of protein-ligand complexes (1702). More specifically, for each protein-ligand complex, the system receives: (i) a respective set of atom features of each atom in the protein and each atom in the ligand, and optionally, (ii) a set of ligand properties of the ligand. For each atom in the complex, the set of atom features of the atom defines at least a 3D spatial position of the atom in a complex where the ligand is bound to a respective binding site on the protein. For each ligand atom in the ligand, the set of atom features of the atom can include additional features such as the hybridization state of the atom, the partial charge of the atom, and the element type of the atom. The set of ligand properties of the ligand can include global ligand properties (that characterize the ligand as a whole rather than being specific to a single atom in the ligand) or atom-specific ligand properties (that characterize specific atoms in the ligand rather than the entire ligand) or both.

The data characterizing the protein-ligand complexes can be obtained, e.g., through physical experiments, or through computational methods (e.g., physics-based simulations or predictions generated by machine learning systems), or through a combination of both.

The system generates a set of training examples (1704). Each training example corresponds to a respective protein-ligand complex and includes data defining: (i) a training input to the design system, and (ii) a target output of the design system.

For each training example, the training input includes protein data and, optionally, a set of ligand design criteria.

The protein data can include any appropriate data characterizing the protein, e.g., data defining one or more amino acid sequences of the protein, or data defining an MSA for the protein, or data characterizing a respective structure of each of one or more template proteins, or a combination thereof.

Optionally, the system can generate ligand design criteria for inclusion in the training input. The ligand design criteria can specify a set of target ligand properties of the ligand, or scaffolding data, or both. The set of target ligand properties can include global ligand properties of the ligand, atom-specific properties of the ligand (e.g., that are derived from the atom features of the atoms in the ligand), or both. The scaffolding data can include ligand scaffolding data that specifies a portion of the 3D structure of the ligand. The system can derive the scaffolding data from the 3D spatial positions of the atoms in the complex as specified by the atom feature data.

The target output of the design system can be based on the atom feature data of the atoms in the ligand, and optionally, of the atoms in the protein. For instance, the target output of the design system can include the atom feature data of each atom in the ligand and of each atom in the protein.

The set of training examples can include training examples with varying amounts of ligand design criteria. For instance, the set of training examples can include some training examples that include no ligand design criteria, some training examples that include ligand design criteria specifying target ligand properties but not scaffolding data, some training examples that include ligand design criteria specifying scaffolding data but not target ligand properties, and some training examples that include ligand design criteria specifying target ligand properties and scaffolding data.

The system can generate multiple training examples corresponding to a single protein-ligand complex, where each training example includes a different set of ligand design criteria based on the known properties of the ligand the known structure of the protein-ligand complex. For instance, for a given protein-ligand complex, the system can generate a first training example that does not include any ligand design criteria, a second training example that includes target ligand properties but not scaffolding data, and a third training example that includes scaffolding data but not target ligand properties.

The system jointly trains the embedding neural network and the generative model on the set of training examples by a machine learning training technique (1706). More specifically, for each training example, the system can process the training input of the training example using the embedding neural network and the generative model to generate a predicted output of the generative model. The system can evaluate an objective function that measures an error (e.g., a root mean square deviation (RMSD), or a mean absolute error (MAE), or a mean squared error (MSE)) between: (i) the predicted output of the generative model, and (ii) the target output of the generative model. The system can determine gradients of the objective function with respect to the parameters of the embedding neural network and the generative model, e.g., using backpropagation. (The parameters of the generative model can include, e.g., a set of neural network parameters of a neural network implemented by the generative model). The system can then update the current values of the parameters of the embedding neural network and the generative model using the gradients, e.g., by the update rule of an appropriate gradient descent optimization algorithm, e.g., RMSprop or Adam.

Specific aspects of the training (e.g., the operations of the generative model during the training and the objective function) may depend on the implementation of the generative model. An example process for training a generative diffusion model that includes a denoising neural network on a training example is described in more detail next with reference to FIG. 14. In the example process of FIG. 14, the operations of the generative diffusion model are modified during training (e.g., as compared to the operations of the generative diffusion model during inference), as will be described in more detail below.

FIG. 14 is a flow diagram of an example process 1400 for jointly training an embedding neural network and a generative diffusion model on a training example. For convenience, the process 1400 will be described as being performed by a system of one or more computers located in one or more locations. For example, a ligand design system, e.g., the ligand design system 100 of FIG. 1, appropriately programmed in accordance with this specification, can perform the process 1400.

The system generates latent conditioning data by processing: (i) the protein data, and (ii) any ligand design criteria, included in the training input of the training example using the embedding neural network (1402).

As part of generating the latent conditioning data, the embedding neural network can generate a design embedding that represents any ligand design criteria provided in the input to the embedding neural network. The design embedding can include a respective atom embedding representing each ligand atom in a set of possible ligand atoms that are eligible for inclusion in the ligand. The number of ligand atoms represented in the design embedding define the maximum number of atoms that the system can select for inclusion in the ligand. As part of generating the latent conditioning data, the system can cause the embedding neural network to generate a design embedding that includes a number of atom embeddings that is at least as great as the number of atoms included in the ligand of the training example.

For each atom in the complex, i.e., for each atom in the protein and for each ligand atom in the set of possible ligand atoms, the system generates target atom state data for the atom based on the set of atom features of the atom (1404). The set of atom features of the atom can include continuous features, e.g., that define the 3D spatial position of the atom and the partial charge of the atom, and/or categorical features, e.g., that define the hybridization state and element type of the atom. For each continuous feature in the atom state data of the atom, the system can populate one or more corresponding dimensions of the target atom state data for the atom with numerical values defining the continuous feature. For each categorical feature in the atom state data, the system can populate a set of corresponding dimensions of the target atoms state data for the atom with a distribution over a set of possible values of the categorical feature, e.g., a one-hot distribution over the set of possible values of the categorical feature that uniquely identifies the actual value of the categorical feature.

If the number of ligand atoms in the set of possible ligand atoms is greater than the actual number of atoms included in the ligand, then the set of possible ligand atoms includes one or more “extra” ligand atoms that will not be selected for inclusion in the ligand. For each extra ligand atom, the system can generate target atom state data that defines the 3D spatial position of the atom as being a predefined “throw-away” position. The system can set the other dimensions of the target atom state data for the extra ligand atom (i.e., the dimensions that do not define the 3D spatial position of the extra ligand atom) to masked (default) values.

The system samples a time step from the sequence of denoising time steps (1406). More specifically, during inference, the generative diffusion model can be configured to perform a sequence of denoising time steps. During training, the system can randomly sample a single denoising time step from the sequence of denoising time steps, e.g., in accordance with a uniform distribution over the sequence of denoising time steps.

The system generates respective noisy atom state data for each atom in the complex by combining random noise with the target atom state data of the atom (1408). For instance, for each atom in the complex, the system can generate the noisy atom state data for the atom by adding random noise to the target atom state data of the atom. The system can scale the random noise combined with the target atom state data of the atoms by a constant that depends on the sampled time step, e.g., where the values of the constants corresponding to the denoising time steps are defined by a noise schedule. Optionally, the system can refrain from combining random noise with any dimensions of the target atom state data for an atom that are designated as being scaffolding data.

The system generates a denoising output using the denoising neural network while the denoising neural network is conditioned on the latent conditioning data (1410). An example process for generating a denoising output is described in detail with reference to FIG. 10. At step 1002 of FIG. 10, the current atom state data for each atom in the complex can be defined as the noisy atom state data for each atom in the complex.

The system determines gradients of an objective function that depends on the denoising output, and uses the gradients to update the parameter values of the denoising neural network and the embedding neural network (1412). The objective function can measure an error between: (i) the denoising output of the denoising neural network, and (ii) a target output of the denoising neural network. The target output of the denoising neural network can define an output of the denoising neural network that, if used to generate a current estimate of the atom state data of the atoms in the complex, would cause the current estimate of the atom state data of the atoms to match the target atom state data of the atoms in the protein-ligand complex of the training example.

In some cases, the dimensions of the target atom state data for the extra ligand atoms (i.e., that are not included in the ligand) other than those that define the 3D spatial position of the extra ligand (i.e., as the throw-away position) are set to masked values. The system can exclude the masked dimensions of the target atom state data for the extra ligand from the computation of the objective function, i.e., such that the objective function is independent of the values of the masked dimensions of the target atom state data.

In general, in implementations of the above described techniques, the protein can be a receptor or enzyme and the ligand for the protein can be an agonist or antagonist of the receptor or enzyme; or the protein can be a cell surface marker and the ligand can be an antibody or aptamer or a label such as a fluorescent label, which binds to the cell surface marker (e.g. for identifying or treating cancerous cells). As another example, the protein can be associated with a disease and the ligand can be an antibody or aptamer marker that binds to, i.e. that recognizes, the protein, for diagnostic purposes. The protein and/or ligand, and in general a molecule or molecule complex as described herein, can be a human, animal, or plant molecule, molecule complex, protein and/or ligand; and any of these can have been derived from human, animal, or plant, e.g. from a sample from a human, animal, or plant.

Some example sources of training data that can be used to train implementations of the system are now described.

In general the training data corresponds to the predictions made and can, for example, include 3D structure data of proteins or ligands, or protein-ligand complexes; examples of bound ligands and/or binding affinity data; molecular property data such as ADMET data where a property is predicted; and so forth.

In general, such data can be obtained by physical experiments and/or using computational methods. Whilst this may initially be slow, an advantage of the described techniques is that, once trained, new information can be obtained quickly, e.g., by generating ligands with high protein-binding affinities.

Also or instead training data can be obtained from a wide range of public or commercial databases. The previously cited papers reference some examples; a few other examples are given below.

For the generative model and the molecular property prediction model, suitable training data can be obtained from, e.g.: ChEMBL (https://www.ebi.ac.uk/chembl/), which is a manually curated database of bioactive molecules with drug-like properties; BioLip (Biological Ligand-Protein Interaction Database), which is a ligand-protein binding database; DUD-E (Directory of Useful Decoys-Enhanced, where decoys are molecules unlikely to bind but with similar physical properties to actives; https://dude.docking.org/); SitesBase (a database for structure-based protein-ligand binding site comparisons).

For molecular properties relating to binding affinity/binding energy, suitable training data can be obtained from, e.g.: PDBbind (experimentally measured binding affinity data (Kd, Ki, IC50) for protein-ligand complexes that are deposited in the PDB) BindingDB; ChEMBL; and ExCAPE-DB.

For other molecular property data, such as ADMET data, suitable training data can be obtained from, e.g.: ChEMBL, PubChem (NCBI, the National Center for Biotechnology Information); or DrugBank. Property data can also be obtained from computational tools such as ADMETlab 2.0, and pkCSM.

Generally, the Protein Data Bank (PDB, wwpdb.org) includes a very large amount of useful data including 3D structures (it could be used to obtain distograms), and chemical and biological information, including via the Chemical Component Dictionary, CCD). Other sources of training data include UniProt (Universal Protein Resource) for protein sequence and functional information; and BRENDA, SABIO-RK (for enzyme information and reaction kinetics.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method performed by one or more computers, the method comprising:
iteratively designing a ligand for binding to a protein by, at each of a plurality of design iterations, performing operations comprising:
generating, by a controller, a current set of ligand design criteria that define desired characteristics of the ligand;
generating, using a generative model and while the generative model is conditioned on: (i) protein data characterizing the protein, and (ii) the ligand design criteria generated by the controller, data defining a set of candidate ligands that are predicted to bind to the protein;
determining, for each of a plurality of the candidate ligands, a respective quality score for the candidate ligand based on properties of the candidate ligand; and
updating the controller using the respective quality scores for the candidate ligands.

2. The method of claim 1, wherein at each of one or more design iterations, updating the controller using the respective quality scores for the candidate ligands comprises:
updating the controller to encourage the controller to generate ligand design criteria that result in generation of candidate ligands with improved quality scores at subsequent design iterations.

3. The method of claim 1, wherein at each of one or more design iterations after a first design iteration, generating, by the controller, the current set of ligand design criteria that define desired characteristics of the ligand comprises:
generating the current set of ligand design criteria based at least in part on quality scores for candidate ligands generated at preceding design iterations.

4. The method of claim 1, wherein over the plurality of design iterations, the controller performs a directed search through a space of possible ligand design criteria to optimize quality scores of candidate ligands generated in accordance with the ligand design criteria.

5. The method of claim 1, wherein the controller is implemented using a policy neural network; and
wherein at each of one or more design iterations, generating, by the controller, the current set of ligand design criteria that define desired characteristics of the ligand comprises:
generating the current set of ligand design criteria as an output of the policy neural network.

6. The method of claim 5, wherein at each of one or more design iterations after a first design iteration, generating the current set of ligand design criteria as an output of the policy neural network comprises:
generating a network input to the policy neural network that includes data representing candidate ligands generated at one or more preceding design iterations; and
processing the network input using the policy neural network to generate the current set of ligand design criteria.

7. The method of claim 6, wherein the network input to the policy neural network comprises, for each candidate ligand selected for inclusion in the network input to the policy neural network, data representing one or more of:
a three-dimensional (3D) structure of the candidate ligand;
a 3D structure of a complex that includes the protein and the candidate ligand; or
a quality score for the candidate ligand.

8. The method of claim 6, wherein generating the network input to the policy neural network comprises selecting a plurality of previously generated candidate ligands for inclusion in the network input by performing operations comprising:
applying a clustering operation to a set of previously generated candidate ligands to identify a set of candidate ligand clusters; and
selecting a plurality of previously generated candidate ligands for inclusion in the network input based on the set of candidate ligand clusters.

9. The method of claim 5, wherein updating the controller using the respective quality scores for the candidate ligands comprises:
training the policy neural network, by a reinforcement learning training technique, using rewards that are based on the quality scores for the candidate ligands.

10. The method of claim 1, wherein the controller is implemented using a genetic algorithm; and
wherein at each of one or more design iterations, generating, by the controller, the current set of ligand design criteria that define desired characteristics of the ligand comprises:
obtaining data defining a current population of ligand design tensors, wherein each ligand design tensor specifies a respective set of ligand design criteria and is associated with an overall quality score that is derived from quality scores of candidate ligands generated in accordance with the set of ligand design criteria;
selecting a set of at least two parent ligand design tensors from the current population of ligand design tensors based on the overall quality scores of the ligand design tensors; and
combining two or more of the parent ligand design tensors to generate a child ligand design tensor that defines the current set of ligand design criteria.

11. The method of claim 10, wherein at each of one or more design iterations, updating the controller using the quality scores for the candidate ligands comprises:
determining an overall quality score for the child ligand design tensor based on the quality scores for the candidate ligands generated based on the child ligand design tensor; and
augmenting the current population of ligand design tensors to include data defining the child ligand design tensor and the overall quality score for the child ligand design tensor.

12. The method of claim 1, wherein the controller is implemented using a simulated annealing algorithm; and
wherein at each of one or more design iterations, generating, by the controller, the current set of ligand design criteria that define desired characteristics of the ligand comprises:
obtaining data defining a current ligand design tensor that defines a set of ligand design criteria; and
generating a candidate ligand design tensor by stochastically modifying the current ligand design tensor, wherein the candidate ligand design tensor defines the current set of ligand design criteria.

13. The method of claim 12, wherein at each of one or more design iterations, updating the controller using the respective quality scores for the candidate ligands comprises:
determining an overall quality score for the candidate ligand design tensor based on the quality scores for the candidate ligands generated based on the candidate ligand design tensor; and
determining whether to update the current ligand design tensor to the candidate ligand design tensor using: (i) a current temperature parameter, and (ii) the overall quality score for the candidate ligand design tensor;
wherein the current temperature parameter at the design iteration is determined based on a predefined schedule.

14. The method of claim 1, further comprising, at each of one or more design iterations, determining a measure of diversity of the set of candidate ligands generated at the design iteration;
wherein the controller is updated at the design iteration based at least in part on the measure of diversity of the set of candidate ligands generated at the design iteration.

15. The method of claim 1, further comprising, at each of one or more design iterations:
generating a plurality of new candidate ligands by fragmenting and reassembling candidate ligands from the set of candidate ligands generated by the generative model at the design iteration; and
augmenting the set of candidate ligands by adding the plurality of new candidate ligands to the set of candidate ligands.

16. The method of claim 1, further comprising, at each of one or more design iterations, generating candidate ligands using the generative model until a termination criterion is satisfied;
wherein the termination criterion is satisfied when a rate of increase in a measure of diversity of the set of candidate ligands generated at the design iteration fails to satisfy a threshold.

17. The method of claim 1, wherein at each of one or more design iterations, the current set of ligand design criteria define a respective target value for each of one or more global properties of the ligand that characterize the ligand as a whole.

18. The method of claim 17, wherein at each of one or more design iterations, the current set of ligand design criteria define a respective target value for a set of global properties of the ligand characterizing one or more of: a binding affinity of the ligand for the protein, absorption properties of the ligand, distribution properties of the ligand, metabolism properties of the ligand, excretion properties of the ligand, toxicity properties of the ligand, a number of rings in the ligand, a molecular weight of the ligand, a lipophilicity of the ligand, an ability of the ligand to donate or accept hydrogen bonds, a total polar surface area of the ligand, a number of rotatable bonds in the ligand, a number of chiral centers in the ligand, a number of electrophilic centers in the ligand, a number of nucleophilic centers in the ligand.

19. A system comprising:
one or more computers; and
one or more storage devices communicatively coupled to the one or more computers, wherein the one or more storage devices store instructions that, when executed by the one or more computers, cause the one or more computers to perform operations comprising:
iteratively designing a ligand for binding to a protein by, at each of a plurality of design iterations, performing operations comprising:
generating, by a controller, a current set of ligand design criteria that define desired characteristics of the ligand;
generating, using a generative model and while the generative model is conditioned on: (i) protein data characterizing the protein, and (ii) the ligand design criteria generated by the controller, data defining a set of candidate ligands that are predicted to bind to the protein;
determining, for each of a plurality of the candidate ligands, a respective quality score for the candidate ligand based on properties of the candidate ligand; and
updating the controller using the respective quality scores for the candidate ligands.

20. One or more non-transitory computer storage media storing instructions that when executed by one or more computers cause the one or more computers to perform operations comprising:
iteratively designing a ligand for binding to a protein by, at each of a plurality of design iterations, performing operations comprising:
generating, by a controller, a current set of ligand design criteria that define desired characteristics of the ligand;
generating, using a generative model and while the generative model is conditioned on: (i) protein data characterizing the protein, and (ii) the ligand design criteria generated by the controller, data defining a set of candidate ligands that are predicted to bind to the protein;
determining, for each of a plurality of the candidate ligands, a respective quality score for the candidate ligand based on properties of the candidate ligand; and
updating the controller using the respective quality scores for the candidate ligands.
